(19) United States
(12) Patent Application Publication
(10) Pub. No.: US 2013/0122589 A1
(43) Pub. Date: May 16, 2013

(54) TARGETED DIFFERENTIATION OF STEM CELLS

(71) Applicants: Susan Kimber, Manchester (GB); Rachel Oldershaw, Hemblington (GB)

(72) Inventors: Susan Kimber, Manchester (GB); Rachel Oldershaw, Hemblington (GB)

(73) Assignee: THE UNIVERSITY OF MANCHESTER, Manchester (GB)

(21) Appl. No.: 13/811,816

(22) PCT Filed: Jul. 26, 2011
(86) PCT No.: PCT/GB2011/051424

§ 371 Date: Jan. 23, 2013

(30) Foreign Application Priority Data
Jul. 26, 2010 (GB) 1012495.6
Jul. 27, 2010 (GB) 1012559.9

(51) Int. Cl. C12N 5/071

(52) U.S. Cl. CPC C12N 5/0655
USPC 435/366

(57) ABSTRACT

The invention provides a method of producing a mesodermal lineage progenitor cell, by 10 application of one or more factors selected from the group consisting of activin, Wnt, BMP, FGF, an inhibitor of activin, GDF and NT to a culture of undifferentiated stem cells for a period of time sufficient to differentiate a stem cell into a mesodermal lineage progenitor cell. The stem cell may be derived from a stem cell line, such as an embryonic stem cell line, for example HUES-1, HUES-7, HUES-8, MAN-1 or MAN-2. Also provided are cell 1 cultures, cells, matrices, kits and uses of the cell cultures and cells.

This invention relates to a directed differentiation protocol to target stem cells toward a particular progenitor phenotype, preferably a progenitor cell phenotype. The protocol uses a series of proteins and growth factors which are applied to the stem cells for a period of time sufficient for their differentiation into a mesodermal lineage progenitor cell, preferably a chondro-, osteo, and/or teno-progenitor cell. Thus, the present invention provides methods for the generation of such a progenitor cell from a stem cell, cell cultures and matrixes comprising cells of the invention, kits for use in the methods of the invention, and the use of the cell cultures and kits in treating bone, tendon and/or cartilage defects in a subject.

### BACKGROUND

Stem cells are undifferentiated cells which have the potential to develop into a range of mature, differentiated cell types. Stem cells can be distinguished based on their differentiation potential. Totipotent stem cells can differentiate into embryonic and extraembryonic cell types, and have the ability to construct a complete, viable, organism. These cells are produced from the fusion of a sperm and an egg. Pluripotent stem cells are the descendents of totipotent stem cells and can differentiate into nearly all the possible cell types of an organism, i.e. the cells derived from the three germ layers, apart from a placenta. Multipotent stem cells can differentiate into a number of cell types, but only those which are closely related. The remaining types of stem cells are unipotent, and thus are more limited in their differentiation capacity, and generally give rise to only specific cell types.

The potential for using stem cells in research and in therapy has been the focus of much attention over recent years. Many of the proposed applications require control of the differentiation pathway of stem cells toward a particular cell type, as and when required.

Human Embryonic Stem (hES) cells have great potential for the generation of cell-based treatments for many diseases because of their properties of pluripotency and limitless self-renewal. However, the realisation of hES cells as a practicable source of cells for clinical use and as therapeutics has been hindered by factors such as the lack of robust and efficient protocols for generating high yields of appropriately differentiated cell types.

Targeted differentiation protocols reported for other cell lineages have exploited knowledge of development, by implementing step-wise differentiation of cells into desired progeny.

The approach of segmenting culture protocols into distinct sequential stages mimicking intermediate differentiation states has been used in the production of insulin-secreting cells, hepatocytes, cardiomyocytes, dopaminergic neurons, and oligodendrocytes. Such methods have formed the basis for improving the efficiency of hES cell differentiation (Murry, C. E. & Keller, G. Differentiation of embryonic stem cells to clinically relevant populations: lessons from embryonic development. Cell 132, 661-680 (2008)).

The development of cartilaginous tissue within hES cell teratomas formed in vivo is readily demonstrated, yet the proportion of chondrocytes obtained from spontaneous differentiation of embryoid bodies (EBs) in vitro is very low (Kawaguchi Bone 36 758-769 2005). Previous protocols have reported ES cell differentiation towards chondrocytes (Kawaguchi et al, supra; Koay et al Stem Cells 25 2183-2190 2007) or multipotent mesenchymal cells (Lian et al Stem Cells 25 425-436 2007) by supplementing the culture medium with pro-mesodermic/pro-chondrogenic growth factors, or by co-culturing hES cells with either chondrocytes or developmentally immature chondroprogenitors. In these protocols the yield of chondrocyte-like cells achieved was limited.

Articular cartilage is vitally important in the joint, providing smooth articulation and sustaining skeletal mobility. Because of its avascular nature, articular cartilage has low intrinsic capacity for repair and is highly susceptible to damage in degenerative conditions, such as osteoarthritis. Joint degeneration with cartilage loss is of high and increasing prevalence and presents a major social and healthcare burden (Goldring et al., J Cell Physiol 213 626-634; Hardingham et al., Oxford Textbook of Rheumatology 325-334 (Oxford University Press 2004)). Whilst joint replacement is successful in the elderly, the lifetime of replacements is too short for younger patients and tissue engineering solutions are being developed to affect a biological repair. These strategies have focused largely on mature articular chondrocytes and adult stem cells as sources of living cells for regenerating cartilage (Hardingham et al., J Anat 209 469-480 (2006) and less attention has been given to human embryonic stem (hES) cells.

### BRIEF SUMMARY OF THE DISCLOSURE

In accordance with the present invention, there is provided in a first aspect a method of producing a mesodermal lineage progenitor cell, the method comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate a stem cell into a mesodermal lineage progenitor cell, preferably a chondro-, osteo-, and/or teno-progenitor cell, preferably a chondro-, osteo-, and/or teno-cyte cell.

Thus, any one or more of activin, Wnt, BMP, FGF, an inhibitor of activin, GDF or NT may be used in a combined, simultaneous or sequential application with any one or more of the remaining factors independently selected from the group consisting of activin, Wnt, BMP, FGF, an inhibitor of activin, GDF and NT.

Thus, activin may be used in the combined, simultaneous or sequential application with one or more factors independently selected from the group consisting of Wnt, BMP, FGF, an inhibitor of activin, GDF and NT.

Wnt may be used in the combined, simultaneous or sequential application with one or more factors independently selected from the group consisting of activin, BMP, FGF, an inhibitor of activin, GDF and NT.

BMP may be used in the combined, simultaneous or sequential application with one or more factors independently selected from the group consisting of activin, Wnt, FGF, an inhibitor of activin, GDF and NT.

FGF may be used in the combined, simultaneous or sequential application with one or more factors independently selected from the group consisting of activin, Wnt, BMP, an inhibitor of activin, GDF and NT.

An inhibitor of activin may be used in the combined, simultaneous or sequential application with one or more factors independently selected from the group consisting of activin, Wnt, BMP, FGF, GDF and NT.

GDF may be used in the combined, simultaneous or sequential application with one or more factors independently selected from the group consisting of activin, Wnt, BMP, FGF, an inhibitor of activin or NT.

NT may be used in the combined, simultaneous or sequential application with one or more factors independently selected from the group consisting of activin, Wnt, BMP, FGF, an inhibitor of activin or GDF.

The application of the factors may be a combined application, a simultaneous application or a sequential application, or any combination or two or more of combined, simultaneous or sequential application. Thus, two or more independently selected factors may be applied in a combined manner, and/or two or more independently selected factors may be applied in a simultaneous manner, and/or one or more independently selected factors may be applied in a sequential manner.

Preferably, the present invention provides for the combined, simultaneous and/or sequential application of one, two, three, four, five, six or seven factors independently selected from the group consisting of activin, Wnt, BMP, FGF, an inhibitor of activin, NT or GDF.

In a preferred embodiment, the first aspect of the invention provides a method of producing a mesodermal lineage progenitor cell from a stem cell, the method comprising i) the combined, simultaneous, and/or sequential application of activin and Wnt to a culture of undifferentiated stem cells for a period of time sufficient to differentiate a stem cell into a mesendoderm cell; followed by ii) the combined, simultaneous, and/or sequential application of one or more factors independently selected from the group consisting of an inhibitor of activin, follistatin and FGF to a culture of cells resulting from i) for a period of time sufficient to differentiate a mesendoderm cell into a mesodermal lineage progenitor cell; optionally followed by iii) the combined, simultaneous, and/or sequential application of GDF and NT to a culture of cells resulting from ii) for a period of time sufficient to differentiate a mesodermal lineage progenitor cell into a chondro-, osteo- and/or teno-progenitor cell.

In a preferred embodiment of the first aspect, the method relates to the production of a chondroprogenitor cell from a stem cell, preferably an embryonic stem cell. Thus, there is provided a method of producing a chondroprogenitor from a stem cell, comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into a chondroprogenitor cell. Preferably, the subsequent passaging is in the presence of one or more factors independently selected from the group consisting of FGF, BMP, an inhibitor of activin, and NT, and optionally GDF.

In a preferred embodiment of the first aspect, the method relates to the production of a osteoprogenitor cell from a stem cell, preferably an embryonic stem cell. Thus, there is provided a method of producing an osteoprogenitor from a stem cell, comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into an osteoprogenitor cell. Preferably, the subsequent passaging is in the presence of one or more factors independently selected from the group consisting of FGF, BMP, an inhibitor of activin, and NT, and optionally GDF.

In a preferred embodiment of the first aspect, the method relates to the production of a tenoprogenitor cell from a stem cell, preferably an embryonic stem cell. Thus, there is provided a method of producing a tenoprogenitor from a stem cell, comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into a tenoprogenitor cell. Preferably, the subsequent passaging is in the presence of one or more factors independently selected from the group consisting of FGF, BMP, an inhibitor of activin, and NT, and optionally GDF.

The subsequent passaging may comprise the addition of one or more factors independently selected from the group consisting of FGF, BMP, an inhibitor of activin, and NT, and optionally GDF either in combination, simultaneously and/or sequentially, or any combination of two or more of combined, simultaneous or sequential application. Thus, two or more independently selected factors may be applied in a combined manner, and/or two or more independently selected factors may be applied in a simultaneous manner, and/or one or more independently selected factors may be applied in a sequential manner.

In a further preferred embodiment, there is provided a method of producing a mesodermal lineage progenitor cell from a stem cell, the method comprising i) the combined, simultaneous, and/or sequential application of one or more factors independently selected from the group consisting of activin, Wnt, FGF and BMP to a culture of undifferentiated stem cells for a period of time sufficient to differentiate the stem cell into a mesendoderm cell; followed by ii) the combined, simultaneous, and/or sequential application of one or more factors independently selected from the group consisting of BMP, an inhibitor of activin, FGF and/or NT to a culture of cells resulting from i) for a period of time sufficient to differentiate a mesendoderm cell into a mesodermal lineage progenitor cell; optionally followed by iii) combined, simultaneous, and/or sequential application of one or more factors independently selected from the group consisting of FGF, BMP, GDF and/or NT to a culture of cells resulting from ii) for a period of time sufficient to differentiate the mesodermal lineage progenitor cell into a chondro-, osteo- and/or teno- progenitor cell.

The present invention also provides a method of producing a chondro-, osteo- and/or teno-progenitor cell from a mesodermal lineage progenitor cell, the method comprising combined, simultaneous, and/or sequential application of one or more factors independently selected from the group consisting of FGF, BMP, GDF and/or NT to a culture of mesodermal lineage progenitor cells. Preferably, the method comprises the application of NT, optionally in combined, simultaneous and/or sequential application of one or more factors independently selected from the group consisting of FGF, BMP and GDF. Optionally, the method may additionally comprise producing the mesodermal lineage progenitor cell from a stem cell, comprising the combined, simultaneous, and/or sequential application of one or more factors independently selected from the group consisting of activin, Wnt, FGF and BMP to a culture of undifferentiated stem cells for a period of time sufficient to differentiate the stem cell into a mesendoderm cell; followed by ii) the combined, simultaneous, and/or sequential application of one or more factors independently selected from the group consisting of BMP, an inhibitor of activin, FGF and/or NT to a culture of cells resulting from i) for a period of time sufficient to differentiate a mesendoderm cell into a mesodermal lineage progenitor cell.

In a further preferred embodiment, there is provided a method of producing a mesodermal lineage progenitor cell from a stem cell, the method comprising, in the following order:

i) the combined, simultaneous and/or sequential application of activin and Wnt to a culture of undifferentiated stem cells;

ii) the combined, simultaneous and/or sequential application of activin, Wnt, and FGF to a culture of cells resulting from step i);

iii) the combined, simultaneous and/or sequential application of activin, Wnt, FGF and BMP to a culture of cells resulting from step ii);

iv) the combined, simultaneous and/or sequential application of FGF, BMP, an inhibitor of activin and NT to a culture of cells resulting from step iii);

v) the combined, simultaneous and/or sequential application of FGF, BMP and NT to a culture of cells resulting from step iv).

Optionally, the method may comprise the following steps to produce a chondro-, osteo-, and/or teno-progenitor cell from a mesodermal lineage progenitor cell;

vi) the combined, simultaneous and/or sequential application of FGF, BMP, GDF and NT to a culture of cells resulting from step v) above;

vii) the combined, simultaneous and/or sequential application of FGF, GDF, and NT to a culture of cells resulting from step vi).

The present invention also provides a method of producing a chondro-, osteo-, and/or teno-progenitor cell from a mesodermal lineage progenitor cell, the method comprising

vi) the combined, simultaneous and/or sequential application of one or more factors independently selected from FGF, BMP, GDF and NT to a culture of mesodermal lineage progenitor cells;

vii) the combined, simultaneous and/or sequential application of one or more factors independently selected from FGF, GDF, and NT to a culture of cells resulting from step vi).

The method may be optionally preceded by any one or more of steps i) to v) above.

Preferably, the time periods for each of the above steps are as follows:

For step i), the preferred time period is 0.5 to 2 days, preferably 1 to 2 days, preferably 1 day;

For step ii), the preferred time period is 0.5 to 4 days, preferably 1 to 3 days, preferably 1 to 2.5 days, preferably 1 to 2 days, preferably 1 day;

For step iii), the preferred time period is 0.5 to 2 days, preferably 1 to 2 days, preferably 1 day;

For step iv), the preferred time period is 3 to 5 days, 3.5 to 4.5 days, preferably 3 to 4 days, preferably 4 days;

For step v), the preferred time period is 0.5 to 2 days, preferably 1 to 2 days, preferably 1 day;

For step vi) the preferred time period is 1 to 4 days, preferably 1.5 to 3.5 days, preferably 2 to 3 days, preferably 2 days; and

For step vii), the preferred time period is 2 to 5 days, preferably 2.5 to 4.5 days, preferably 3 to 4 days, preferably 3 days.

During, or by the end of the steps mentioned above, the following markers may be observed:

| STEP | One or more markers selected from the group consisting of:- | |
|---|---|---|
| i | Oct 4 | |
| | Nanog | |
| | MixL (low) | |
| | Bra | |
| | GSc | |
| | Wnt 3 | |
| | N-cad | |
| | E-cad | |
| | Sox 17 (low) | |
| ii | Oct 4 | |
| | MixL (low but preferably higher than in i) | |
| | Bra (high) | |
| | GSc | |
| | E-cad | |
| | Gata 4 | |
| | Sox 17 | |
| iii | As for step ii but preferably lower GSc and/or Gata 4 | |
| iv | low or absent: | Oct 4 |
| | | Nanog |
| | | Gata 4 |
| | | Sox 17 |
| | | Sox 1 |
| | | Pax 6 |
| | | E-cad |
| | Presence of: | MixL, |
| | | PDGF-Rβ |
| | | Flk 1 |
| | | Sox 9 |
| | | Bra (low) |
| v | As for step iv but low markers are lower, high markers are | |
| | higher. | |
| | Absence of Bra. | |
| vi | Low or absent: | Nanog |
| | | Oct 4 |
| | | Sox 2 |
| | | E-cad |
| | | Gata 4 |
| | | Sox 17 |
| | | Bra |
| | Presence of: | Sox 9 |
| | | Collagen 2 |
| | | Sox 6 |
| | | CD44 aggrecan |
| | Absence of | Flk 1 |

The above markers serve as a guide only to indicate the passage of the cells from one step to the next. In a preferred embodiment, each step is carried out for a time period sufficient for the cells to exhibit expression and/or absence of one or more of the markers selected from the groups listed above.

Another guide in determining the passage of the cells through the steps from a stem cell to a chondro, teno and/or osteo-progenitor cell is cell morphology. In a preferred embodiment, each step is carried out for a time period sufficient for the cells to exhibit the following morphological characteristics:

Step i ES like

Step ii Similar to i)

Step iii Initial mesenchymal characteristics

Step iv Initial fibroblastic characteristics, beginning to form whorls

Step v Initial fibroblastic characteristics beginning to form clumps

Step vi Clump formation

Step vii Clear aggregates of rounded cells with few cells in between.

In a preferred embodiment, the first stage is performed for a time period sufficient for the resulting mesendoderm cells to show expression of MIXL1, preferably said expression being higher than the expression in the originating stem cells. Preferably, the second stage is for a time period sufficient for the mesodermal lineage progenitor cells to show expression of brachyury, preferably an increase in expression compared to the mesendoderm cells, and/or Sox9, again preferably an increase in expression compared to the mesendoderm cells. Preferably, the optional third stage is performed for a time period sufficient for the chondro-, osteo-, and/or teno-progenitor cells to show expression of MixL 1 and PDGF-Rβ compared to the mesodermal lineage progenitor cells from which they originate.

Whilst generally speaking, a “day” in the context of the present invention is a continuous 24 hour period, a skilled person will appreciate that time periods defined herein as a day or part of a day may be extended or reduced whilst still achieving the invention. For example, any of the specified time periods may be extended or reduced by up to 25%, for example 20%, 15%, 10%, 9%, 8%, 7%, 6%, 5%, 4%, 3%, 2% or 1%. These ranges apply to both individual time periods for any particular stage or step of the method, and to the overall time period for the method to be carried out.

Preferably, the method of the invention comprises further differentiating any chondroprogenitor, osteo-progenitor and/or teno-progenitor cells. In a preferred embodiment, the method may provide for differentiating the chondroprogenitor cells into chondrocyte cells, the osteoprogenitor cells into osteocyte cells and/or the tenoprogenitor cells into tenocyte cells.

Preferably, in the methods of the invention, the cells are grown without the use of feeder cells. Preferably, the cells are also grown in a serum-free culture medium.

There is provided in a second aspect of the invention the use of one or more factors selected from the group consisting of activin, Wnt, an inhibitor of activin, BMP, FGF, GDF and NT in the combined, simultaneous, and/or sequential application to a culture of undifferentiated stem cells for a period of time sufficient to differentiate a stem cell into a mesodermal lineage progenitor cell, and preferably into a chondro-, osteo- and/or teno-progenitor cell, and more preferably a chondro-, osteo-, and/or teno-cyte cell.

There is provided in a third aspect of the invention, a cell culture produced during or by a method of the present invention. The cell culture may comprise differentiated chondro-, osteo-, and/or teno-progenitor cells.

In a preferred embodiment of the third aspect, there is provided one or more cell cultures selected from the group consisting of:

a cell culture comprising a) undifferentiated stem cells, and b) one or more factors independently selected from the group consisting of activin, Wnt, FGF, and BMP;

a cell culture comprising a) undifferentiated stem cells and mesendoderm cells; and b) one or more factors independently selected from the group consisting of activin, Wnt, FGF, and BMP;

a cell culture comprising a) mesendoderm cells; and b) one or more factors independently selected from the group consisting of BMP, FGF, NT and an inhibitor of activin;

a cell culture comprising a) mesendoderm and mesodermal lineage progenitor cell; and b) one or more factors independently selected from the group consisting of BMP, FGF, NT and an inhibitor of activin;

a cell culture comprising a) mesodermal lineage progenitor cells; and b) one or more factors independently selected from the group consisting of FGF, BMP, GDF and NT;

a cell culture comprising a) mesodermal lineage progenitor cells, chondro-, osteo- and/or teno- progenitor cells; and b) one or more factors independently selected from the group consisting of FGF, BMP, GDF and NT; and

a cell culture comprising a) a chondro-, osteo-, and/or teno-progenitor cell, and b) one or more factors independently selected from the group consisting of FGF, BMP, an inhibitor of activin, NT and GDF; and a chondro-, osteo-, and/or teno-cyte cell.

There is provided in a fourth aspect of the present invention a cell culture according to the third aspect for use in the treatment of a bone, tendon and/or cartilage defect in a subject.

There is provided in a fifth aspect of the invention, a cell produced by a method of the invention. Preferably, the cell is a cell independently selected from the group consisting of: a mesendoderm cell, a mesodermal lineage progenitor cell, a mesoderm cell, a chondro-osteo-, and/or teno-progenitor cell, and a chondro-, osteo-, and/or teno-cyte cell. Preferably, a cell produced by a method of the invention will exhibit reduced levels of one or more markers independently selected from the group consisting of Coll II, PDGFRβ, and/or sulphonated GAGs compared to a native cell of the same type. The cells will preferably show a morphological characteristic disclosed above. In addition, aggregates may be slightly translucent.

There is provided in a sixth aspect, a matrix comprising one or more cells of the invention.

There is provided in a seventh aspect of the invention, a kit of parts comprising, in separate containers, one or more factors independently selected from the group consisting of activin, Wnt, BMP, FGF, an inhibitor of activin, GDF, NT and a culture of undifferentiated stem cells. For combined application, two or more of the above factors may be provided in a combined preparation. Also provided in the kit may be instructions for use, and/or a protocol detailing the method of the invention.

### BRIEF DESCRIPTION OF THE DRAWINGS

Embodiments of the invention are described hereinafter with reference to the accompanying drawings, in which:

FIG. 1 is a schematic of directed differentiation protocol. The protocol is divided into 3 stages. In stage 1 pluripotent hES cells are directed towards a bi-potent mesendoderm population, in stage 2 differentiation proceeds to a mesoderm population and in stage 3 towards chondrocytes. Since some genes are known to be expressed in different cell lineages and at different stages, the developmental status of each cell population was characterized by expression of panels of marker genes including SOX2, which is expressed by both pluripotent hES cells as well as cells derived from the neurectoderm germ layer; ECAD is expressed on pluripotent and mesendoderm cells and CXCR4 has been used to characterize cell lineages from both the endoderm and mesodermal-derived hemangioblast.

FIG. 2 shows the morphology of hES cell cultures (HUES1) at different stages of the protocol. (a, b) Pluripotent hES cells on a MEF feeder layer. Cell cultures were heterogeneous with hES cells forming individual, tightly-packed colonies. (c, d) Pluripotent hES cells cultured on a fibronectin matrix in a defined medium. hES cells appeared as a homogeneous 2D monolayer with individual cells appearing larger than those in colonies maintained on feeder layers. The cells had characteristic hES cell morphology, with a high nucleus to cytoplasm ratio and prominent nucleoli. (e, f) At the end of Stage 1 the cells were approximately 80% confluent and still retained the morphological features of pluripotent hES cells. (g, h) At Stage 2 cell cultures were densely-packed and with phase-bright cell clusters distributed throughout the culture (circled). These cell clusters formed 3D nodules with cells clustered into a “rosette-like” morphology (circled). (i, j) During Stage 3 (day 12) the flatter cells surrounding the 3D cell aggregates began to detach leaving behind the 3D cell aggregates (circled). Note these cultures are on fibronectin:gelatin substrate and were imaged immediately prior to passaging onto gelatin as detailed in the protocol described in Table 1. (k, l) By the completion of Stage 3 (day 14) intermediate cells between the cell aggregates had also become detached. All scale bars=100 μm.

FIG. 3 shows gene expression analysis hES cells at different stages of the protocol. Pluripotent hES cells (HUES1) (grey bars) and differentiating cultures at the end of each stage 1-3 (black bars) were analysed for gene expression, as denoted in FIG. 1. (a-d) genes associated with hES cell pluripotency, (d-f) genes expressed by cell lineages from the mesendoderm, (g-k) genes expressed by mesodermal cell types, (k-o) genes expressed endoderm cell types, (c, p-r) genes expressed by neurectodermal cell types, (s-x) genes expressed by chondrocytes. Results showed that hES cells transiently expressed genes associated with a mesendoderm phenotype, prior to expression of genes associated with mesodermal cell lineages. At the end of the protocol the cells had minimal expression of genes associated with pluripotency, developmental intermediates and non-target cell lineages. FGF5, PAX6 and SOX1, associated with neurectoderm were expressed at an extremely low level in pluripotent hES cell cultures and during all stages of culture in the protocol. Therefore embryoid body-derived spontaneous differentiation cultures (SDC) (hatched bars) taken at day 14) were used as a positive control in order to confirm the specificity of the primers used and to verify the pluripotent phenotype of the original hES cell cultures. Gene expression was normalized to GAPDH. Values represent means±SEM (N=4). *, P<0.05, **, P<0.01, ***, P<0.001.

FIG. 4 shows characterization of sulphated glycosaminoglycan accumulation in cell cultures during directed differentiation of hES cells (HUES1) to chondrocytes. (a, b) Cell clusters that formed throughout Stage 2 cultures stained discretely with safranin O indicative of the accumulation of sGAG. (c, d) At Stage 3 independent cell aggregates were larger than the cell clusters in Stage 2 and showed markedly stronger staining for safranin O. The frequency of safranin O positive cell clusters in Stage 2 cultures and cell aggregates in Stage 3 cultures was determined as in the Methods section. Values represent means±SEM (N=4). To demonstrate the specificity of safranin O staining for sGAG, some cultures were treated with chondroitinase ABC before staining. Control Stage 3 cultures prior to (e) and after (f) staining with safranin O. Stage 3 cultures which had been treated with chondroitinase ABC prior to (g) and after (h) staining with safranin O. Images show that safranin O staining is much reduced in cultures which had been treated with chondroitinase ABC indicating that safranin O staining was specific to sGAG. All scale bars=100 μm. (i) Quantification of sGAG production per cell. Stage 2 and Stage 3 cultures were digested with papain and the amount of sGAG produced per cell quantified by 1,9-dimethylmethylene blue (DMMB) assay normalized against the amount of DNA in each culture. Values represent means±EM (N=4). *, P<0.05.

FIG. 5 shows immunofluorescence of SOX9 and COLLAGEN II. Proteins were indirectly labelled with secondary Alexa Fluor® 488 antibodies (green channel) and cell nuclei labelled with DAPI (blue channel). (a-c) expression of chondrogenic transcription factor SOX9 was low in pluripotent hES cell cultures (HUES1). (d-i) at the end of the differentiation protocol SOX9 was highly expressed and protein was localized within the nucleus of cells within the aggregate cells. (j-p) Phase images and immunofluorescence of COLLAGEN II protein deposited within the matrix of cell aggregates at the end of the differentiation protocol. Scale bar=100 μm.

FIG. 6 shows flow cytometry analysis of HUES1-derived cells at the end of Stage 3. Cells were analysed for expression of the chondrocyte transcription factor SOX9, the cell surface receptor CD44 and the adult stem cell surface antigen CD105. (a) Immunological control for (b) the proportion of SOX9-expressing cells was 74.8%. +/−3.0% (c) Immunological control for (d) the proportion of CD44-expressing cells was 34.1%. +/−2.1% (e) Immunological control for (f) the proportion of CD105-expressing cells (12.7% +/−2.5%). Values represent means±SEM (N=4).

FIG. 7 shows cell culture (HUES1) expansion during directed differentiation of hES cells. The total number of cells within cultures undergoing the directed differentiation protocol was determined on the days on which passaging was carried out, as denoted in Table 1. During directed differentiation the total number of cells within the culture increased by 8.5-fold. Values represent means±SEM (N=4). *, P<0.05.

FIG. 8 shows immunofluorescence of proteins expressed in Stage 1 of directed differentiation and quantification of E-CADHERIN expression by flow cytometry. Pluripotent hES cells and Stage 1 directed differentiation cultures (HUES1) were analysed by immunofluorescence for expression of BRACHYURY, GOOSECOID and E-CADHERIN. Proteins were indirectly labeled with secondary Alexa Fluor® 488 antibodies (green channel) and cell nuclei labeled with DAPI (blue channel). Pluripotent hES cells expressed low levels of BRACHYURY and GOOSECOID though both of these proteins were upregulated and expressed in over 90% of the cell culture at Stage 1. Analysis of E-CADHERIN expression by flow cytometry and immunofluorescence showed that the proportion of E-CADHERIN positive cells was unchanged during the first 3 days of directed differentiation though there were areas of the cell culture in which immunofluorescence were stronger as revealed by immunofluoresecence. Scale bar=100 μm. Values represent means±SEM (N=4).

FIG. 9 shows regulation of genes expressed by primitive hepatocyte cell lineages during directed differentiation of hES cells. Pluripotent hES cells (HUES1) (grey bars) directed differentiation cultures at the end of each stage 1-3 (black bars), embryoid body-derived spontaneously differentiating cells (SDC) at day 14 of culture (broken hatched) bars and fetal liver cDNA (clear bars) were analysed for expression of genes associated with differentiation toward primitive hepatocyte cell lineages, HNF1a, HNF4a, PROX1, ALB and AFP. These genes were expressed at such low levels at all stages of our directed differentiation protocol that embryoid body-derived spontaneously differentiating cultures and fetal liver cDNA were used as positive controls for primer efficacy. Gene expression was normalized to GAPDH. Values represent means±SEM (N=4). *, P<0.05, **, P<0.01, ***, P<0.001.

FIG. 10 shows flow cytometry of CD105 cell surface antigen on undifferentiated hMSC and COL10A1 gene expression analysis during chondrogenic differentiation of hMSC in comparison to directed differentiation of hES cells to chondrocytes. hMSC were derived from human bone marrow mononuclear cells and placed into chondrogenic cell aggregate culture as described in the Supplementary Methods. RNA extracts at day 0 (immediately after being placed into cell aggregate culture), 1, 7, and 14 were analyzed for gene expression of COLLAGEN II and COLLAGEN X. Note the concurrent upregulation of COLLAGEN II and COLLAGEN X from day 7 onwards. In contrast, there was no significant expression of COLLAGEN X during directed differentiation of hES cells (HUES1) indicating that the chondrocytes generated at the end of the directed differentiation protocol did not have a hypertrophic phenotype. Gene expression levels were normalized to GAPDH and values represent mean±SEM (N=3). Analysis by flow cytometry at passage 3 shows that the undifferentiated hMSC are positive for the cell surface antigen CD105.

FIG. 11 shows regulation of genes expressed by related mesodermal cell lineages during directed differentiation of hES cells to chondrocytes. Pluripotent hES cells (HUES1) (grey bars) and differentiating cultures at the end of each stage 1-3 (black bars) were analysed for expression of CBFA1 (osteoblast), PPARγ (adipocyte) and SCLERAXIS (tenocyte). CBFA1 showed the most evidence of gene regulation, increasing in expression between Stage 1 and Stage 2 before being down regulated at Stage 3. PPARγ showed a very slight increase between Stage 2 and Stage 3 whereas SCLERAXIS was barely expressed. Gene expression was normalized to GAPDH. Values represent means±SEM (N=4). *, P<0.05, **, P<0.01.

FIG. 12 shows immunofluorescence of Stage 3 cell cultures at the end of HUES1 directed differentiation. Stage 3 cultures at the end of the directed differentiation protocol were analysed by immunofluorescence. Proteins were indirectly labeled with secondary Alexa Fluor® 488 antibodies (green channel) and cell nuclei labeled with DAPI (blue channel). These cultures were negative for OCT4, NANOG and SOX2 indicative of the loss of pluripotency. In contrast to embryoid body outgrowths these cultures showed negligible expression of proteins expressed by developmental intermediate populations and non-target cell types as there was minimal expression of MIXL1 (mesendoderm), BRACHYURY (early mesoderm), PDFGRβ and FLK1 (mesodermal hemangioblast), GATA4, FOXA2, SOX17, SOX7 (endoderm) and SOX1 and SOX2 (neurectoderm). We did detect expression of PDGRFβ (late mesoderm) and CD44 (chondrogenic). Scale bar=100 μm.

FIG. 13 shows immunofluorescence of proteins expressed in embryoid body unspecified spontaneous differentiation cultures. Proteins were indirectly labeled with secondary Alexa Fluor® 488 antibodies (green channel) and cell nuclei libeled with DAPI (blue channel). Un-directed spontaneous differentiating cultures (outgrowths of HUES1 embryoid bodies which had been cultured for 14 days) were analysed by immunofluorescence to show the heterogeneous nature of the differentiation contrasting with the directed protocol. These cultures were negative for the transcription factors OCT4 and NANOG indicating the loss of pluripotency within the cultures. The pluripotent phenotype of the hES cells was reflected by the expression of proteins associated with the three embryonic germ layers: SOX2 and SOX1 (neurectoderm), GATA4, FOXA2, SOX17 and SOX7 (endoderm), brachyury, PDGFRβ, PDGFRβ and FLK1 (mesoderm). Proteins typically expressed both transiently and during early differentiation (MIXL1, Brachyury, FOXA2 and SOX17) were expressed by approximately 5-10% of the population. Of the chondrocyte-associated, proteins, SOX9 expression was observed within occasional areas of the embryoid body cultures; but was frequently cytoplasmic and rarely nuclear. Pan-CD44 localization was present within some embryoid body cells with punctate immunostaining at the cell surface. Only one area was observed as showing weakly positive immunofluorescence for collagen type II in a few cells within the embryoid body outgrowths.

FIG. 14 shows flow cytometry analysis of SOX9 protein at the end of directed differentiation of hES cell lines HUES7 and HUES8. Directed differentiation was carried out on hES cell lines HUES7 and HUES8 and cell progeny within the gated population shown were analysed for expression of the chondrocyte transcription factor SOX9 in parallel to a negative immunological control. Flow cytometry analysis revealed that 95%±1.01 and 97%±0.3 of cell progeny differentiated from HUES7 and HUES8 respectively were positive for SOX9 protein. Values represent means±SEM (N=4).

FIG. 15 shows the amino acid sequences of of activin, Wnt, BMP, FGF, follistatin, GDF, and NT.

### DETAILED DESCRIPTION

In a first aspect of the present invention, there is provided a method of producing a mesodermal lineage progenitor cell, comprising the combined, simultaneous or sequential application of one or more independently selected growth factors to a stem cell for a period of time sufficient to differentiate the stem cell into a mesodermal lineage progenitor cell. Optionally, the mesodermal lineage progenitor cell may then be differentiated into a chondro-, osteo-, and/or teno-progenitor cell by the combined, simultaneous or sequential application of one or more independently selected growth factors for a period of time sufficient for said differentiation to occur. Preferably, the method optionally includes differentiating the progenitor cells to chondro-, osteo- and/or teno-cyte cells.

The method comprises the combined, simultaneous and/or sequential application of one or more factors independently selected from the group consisting of activin, Wnt, BMP, FGF, an inhibitor of activin, GDF, and NT to a culture of undifferentiated stem cells to produce a mesodermal lineage progenitor cell, after a sufficient period of time.

In a preferred embodiment, there is provided a method of producing a mesodermal lineage cell from a stem cell, the method comprising i) the combined, simultaneous, and/or sequential application of one or more factors independently selected from the group consisting of activin, Wnt, FGF and BMP to a culture of undifferentiated stem cells for a period of time sufficient to differentiate the stem cell into a mesendoderm cell; followed by ii) the combined, simultaneous, and/or sequential application of one or more factors independently selected from the group consisting of BMP, an inhibitor of activin, FGF and/or NT to the culture of cells resulting from of i) for a period of time sufficient to differentiate the mesendoderm cell into a mesodermal lineage progenitor cell; optionally followed by iii) the combined, simultaneous, and/or sequential application of one or more factors independently selected from the group consisting of FGF, BMP, GDF and/or NT to the culture of mesodermal lineage cells resulting from ii) for a period of time sufficient to differentiate the mesodermal lineage progenitor cells into a chondro-, osteo- and/or teno-progenitor cell.

In the present invention, “combined” application of two or more independently selected factors means that the two or more factors are combined (e.g. mixed together), prior to application to the cells. By “simultaneous” application is meant that two or more of the independently selected factors are applied to the cells, separately but at substantially the same time. Thus, the factors are provided in separate containers, but applied to the cells at the same time. By “sequential” application, means that one or more factors or combined factors are applied to the cells in turn, separated in a temporal manner. The present invention allows the application of one or more independently selected growth factors selected from activin, Wnt, BMP, FGF, an inhibitor of activin, GDF and NT in a combined manner, a simultaneous manner or a sequential manner. It also allows for any combination of two or more of combined, simultaneous or sequential application of one or more independently selected growth factors selected from activin, Wnt, BMP, FGF, an inhibitor of activin, GDF and NT. This means that two or more independently selected factors may be combined, and applied, and/or two or more independently selected factors may be simultaneously applied, and/or two or more independently selected factors may be sequentially applied. In the present invention, the combined, simultaneous and sequential application may take place in any preferred order.

The stem cells used in the present invention are preferably embryonic stem cells. Preferably, they are derived from a pre-implantation stage embryo or a pen-implantation stage embryo. Most preferred are embryonic stem cells derived from an epiblast, post implantation. Such stem cells are known as epistem cells. Also included are primordial germ cells (hEG). Cells for use in the invention may include any cells taken within 6 days, post-fertilisation. Preferably, the stem cells used in the invention are karyo-typically normal and not derived from a malignant source. The stem cells may be pluripotent or totipotent, preferably the former. The stem cells may be derived directly from tissue, or from an established cell line. A cell line is a population of cells that can be propagated in culture through at least 10 passages. Examples of suitable cell lines include HUES-1, HUES-7, HUES-8, MAN-1 and MAN-2 (See Cavan et al New Engl J Med 350, 1353-1356 (2004)). Each of these cell lines has been deposited with the UK Stem Cell Bank, National Institute for Biological Standards and Control, Blanche Lane, Potters Bar, Hertfordshire, EN6 3QG, under Accession No.s P-10-009 (Man1) and P-10-010 (Man 2). Further details and characterisation of man1 and Man2 cell lines are provided below, and in Camarasa et al (In Vitro Cell. Dev. Biol. Animal (2010) 46:386-394). Where cell lines are not used but the cells are derived directly from an embryo, then preparation of the embryo may be performed as described in Camarasa et al (In Vitro Cell. Dev. Biol. Animal (2010) 46:386-394). In certain aspects of the invention, the stem cells may be derived from a donor subject, to whom the cells are re-administered after undergoing the methods of the invention. Preferably, the population of stem cells will be homogenous.

Man1 is derived from Human blastocyst tissue at day 7 of development. The tissue of origin was fresh. The cells form colonies and are pluripotent, as shown by Immunofluorescence protocol to detect common pluripotency markers using commercial antibodies (R&D). The cell line spontaneously differentiates in vitro to EBs upon fetal bovine serum addition, which grew and differentiated into cells of the three germ layers, as detected by immunofluorescence. The following markers can be used to characterise the cell line: Nanog, Oct-4, SSEA-3, TRA-1-80, hTERT, all positive. and SSEA-1 negative, at passages 8-10.

Man2 cells were derived from chemically activated clinically failed to fertilise oocyte, cultured to day 6 blastocyst, graded 6Aa. The tissue of origin was fresh. The cells form colonies and are pluripotent, as shown by Immunofluorescence protocol to detect common pluripotency markers using commercial antibodies (R&D). The cell line spontaneously differentiates in vitro to EBs upon fetal bovine serum addition, which grew and differentiated into cells of the three germ layers, as detected by immunofluorescence. The following markers can be used to characterise the cell line: Nanog, Oct-4, SSEA-3, TRA-1-80, hTERT, all positive. SSEA-1 negative at passage 12.

Their karyotypic stability has been confirmed by standard G banding. Both lines showed an apparently normal diploid female karyotype at passages 38 and 22, respectively, after a year and 7 mo in continuous culture. As both lines are female, a DNA profile was carried out to rule out the possibility of cell culture contamination/mixing between them, which resulted in allele patterns corresponding to unique identity from different individuals, differences being detected at 14 out of the 15 loci examined

TABLE A
List of differentiation markers detected in Man-1 and Man-2 after embryoid body formation
for 10 d and adherent culture for 28 to 34 d

| Ectoderm | | | Mesoderm | | | Endoderm/Ectoderm | | |
|---|---|---|---|---|---|---|---|---|
| | Man-1 | Man-2 | | Man-1 | Man-2 | | Man-1 | Man-2 |
| GFAP | − | − | ASMA | + | + | AFP | + | + |
| β-tub III | + | + | Aggrecan | + | − | Cdx2 | − | + |
| NeuroD1 | − | + | Brachyury | + | + | GATA4 | nd | + |
| NeuroD2 | nd | − | CD31 | + | Nd | Pax4 | nd | + |
| Neurog 3 | + | − | Collagen II | + | + | Pax6 | nd | + |
| Musashi-1 | nd | + | Jagged-1 | Nd | − | Pdx-1 | − | − |
| | | | Vimentin | + | + | SOX17 | nd | − |

TABLE B
Origin and characterisation of Man-1 and man-2 hESC lines

| | Man-1 | Man-2 |
|---|---|---|
| Origin | Fresh supernumery | Failed to fertilise |
| | embryo | embryo |
| Paternally imprinted | H19+ | H19+ |
| gene expression | SNRPN+ | SNRPN+ |
| | IGF2+ | IGF2+ |
| Derivation stage | d + 7 | d + 6 |
| Stem cell markers | Nanog, Oct-4, TRA-1- | Nanog, Oct-4, TRA-1- |
| | 60, TRA-1-81, SSEA- | 60, TRA-1-81, SSEA-3, |
| | 3, SSEA-4 | SSEA-4 |
| In vitro differentiation | 3 germ layers | 3 germ layers |
| Karyotype | 46, XX | 46, XX |
| In vivo differentiation | In progress | yes |
| (teratomas formation) | | |

Preferred cell lines for use in the invention are those which have one or more of the following characteristics: a) are derived from embryos, preferably embryonic stem cells, preferably at derivation stage d+6 or d+7 or later, b) have a karyotype of 46, c) exhibit paternally imprinted gene expression of H19+, SNRPN+ and/or IGF2+, and d) exhibit stem cell markers Nanog, Oct-4, TRA-1-60, TRA-1-81, SSEA-3, and/or SSEA-4.

Preferably, the embryonic stem cells used in the invention are capable of being maintained in an undifferentiated state in vitro when cultured with or without feeder cells under non-differentiating conditions.

By “undifferentiated” is meant that the stem cell has not begun commitment to any particular cell lineage. Such cells typically display the morphological characteristics of undifferentiated cells, which distinguish them from differentiated cells. Such morphological characteristics include the presence of high nuclear/cytoplasmic ratios and prominent nucleoli. A cell population which is substantially undifferentiated will comprise at least 50%, 60%, 70%, 80%, 90%, 95%, 96%, 97%, 98%, 99% or 100% undifferentiated stem cells. In the context of the present invention, an undifferentiated stem cell population refers to the starting population of method.

Animal sources of stem cells for use in the invention include any mammal, preferably primates, mice, rats, horses or humans. Most preferred are human stem cells, preferably human embryonic stem cells, preferably human embryonic pluripotent stem cells. Most preferably, an established cell line is used, for example a cell line as described herein.

In the embodiments of the invention where the stem cells are derived from tissue, this may be performed using methodology available in the art. For example, hES cells can be prepared as described by Thomson et al (U.S. Pat. No. 5,843,780; Science 282:1145, 1998; Curr. Top. Dev. Biol. 38:133 ff., 1998; Proc. Natl. Acad. Sci. USA 92:7844; 1995).

Human Embryonic Germ (hEG) cells can be prepared from primordial germ cells present in human foetal material taken about 8-11 weeks after the last menstrual period. Suitable preparation methods are described in Shamblott et al., (Proc. Natl. Acad. Sci. USA 95:13726, 1998 and U.S. Pat. No. 6,090,622).

The invention further encompasses the differentiation of chondro, osteo and/or teno progenitor cells to chondrocytes, osteocytes and/or tenocytes. This may be achieved by the addition of one or more growth factors, selected from the group consisting of FGF, BMP, GDF and NT.

The factors used to direct differentiation of the stem cell to a mesodermal lineage progenitor cell may be applied to the stem cells or progeny thereof in combination, sequentially or simultaneously. Thus, preferably the method of the invention is a multi-stage process in which one or more factors is applied to the culture for a period of time sufficient for a cell to reach a pre-defined stage of differentiation. Once such a level of differentiation has been achieved by a pre-defined proportion of the cells in the culture, the next stage of the method is commenced in which one or more factors of the previous stage may continue to be applied to the culture, whilst one or more other factors may be inhibited, removed or no longer applied, and one or more different factors may be applied. Thus, where the invention is a multi-stage process, the method allows for overlap between the stages in terms of the factors being applied to the culture. If applied sequentially, then two or more factors may be applied in combination or simultaneously, sequentially to one or more other factors. Thus, the methods allow for the application of one or more factors to be applied in combination, and then the application of one or more of these factors to be stopped and the application of one or more different factors to be initiated. Each factor may be applied for all or part of each stage of the method. In addition, the method in each stage allows for the culturing of the cells for a period of time for differentiation to occur, during which no factors are applied to the culture.

The starting point for the protocol of the present invention is preferably stem cells, as defined herein. However, it is also envisaged that the invention may be achieved using cells which have begun differentiation towards a chondro-, osteo-, and/or teno-cyte cell lineage. In such an embodiment of the present invention, one or more of the appropriate lineages may then be applied to the cells to target differentiation towards a mesodermal lineage progenitor cell type, or optionally further differentiation towards a chondro-, osteo-, and/or teno-progenitor cell type, or chondro-, osteo-, and/or teno-cyte cell type. Thus, for example where the starting cells are mesendoderm cells, the method for differentiation may comprise the combined, simultaneous and/or sequential application of one or more factors selected from the group consisting of BNP, an inhibitor of activin, FGF and/or NT to the mesendoderm cells for a period of time sufficient to differentiate the mesendoderm cells into a mesodermal lineage progenitor cell. Where the starting cells are mesodermal lineage progenitor cells, the protocol may comprise the combined, simultaneous and/or sequential application of one or more factors selected from the group consisting of FGF, BNP, GDF and/or NT to the culture of cells for a period of time sufficient to differentiate the mesodermal lineage progenitor cells into a chondro-, osteo-, and/or teno-progenitor cell.

The time taken for a stem cell to differentiate into a mesodermal lineage progenitor cell is typically measured in units of days, although it is possible that units of hours may be used. Each factor may be applied once or repeatedly over a period of time. Where application is repeated, this may be at any suitable time interval. Preferably, repeated application is performed at daily time intervals.

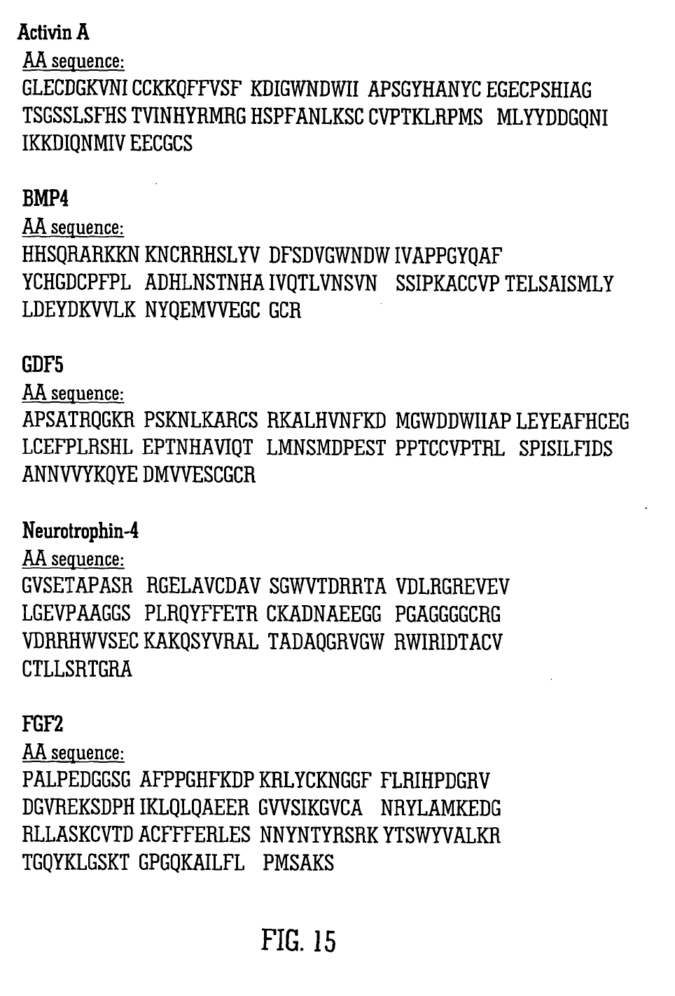

The invention of the method comprises the application of activin to a culture of undifferentiated stem cells. Activin is a protein dimer, which plays a role in cell proliferation and differentiation. Activin includes any protein or polypeptide having activin biological activity, such as the ability to promote proliferation and differentiation. Included are isolated, purified and/or recombinant forms of activin, functional derivatives and homologues, or fragments of activin which retain activin's ability to promote proliferation and differentiation. In a preferred embodiment, activin has the amino acid sequence of FIG. 15. Functional derivatives and homologues may share at least 60%, 65%, 70%, 75%, 80%, 85%, 90%, 91%, 92%, 93%, 94%, 95%, 96%, 97%, 98%, or 99% sequence identity with the wild type activin sequence, for example as shown in FIG. 15, over the whole of the sequence or a fragment thereof. Fragments of activin useful in the present invention may be at least 50%, 55%, 60%, 65%, 70%, 75%, 80%, 85%, 90%, 91%, 92%, 93%, 94%, 95%, 96%, 97%, 98%, or 99% the length of the full length activin sequence, for example as shown in FIG. 15.

Preferably, activin is applied in decreasing amounts, preferably over the period of time taken for differentiation of a stem cell into a mesendoderm cell. Preferably, multiple doses of activin are applied, preferably at daily intervals. Preferably, activin is initially applied at a concentration of between 30 to 120 ng/ml, preferably 40 to 60 ng/ml, more preferably 45, 50, 51, 52, 53, 54, 55, 56, 57, 58, 59 or 60 ng/ml. The amount of activin may then be decreased to between 10 and 35 ng/ml, preferably between 15 and 30 ng/ml, preferably 20, 21, 22, 23, 24, 25, 26, 27, 28, 29 or 30 ng/ml. Finally, the amount of activin is decreased to between 1 and 2Ong/ml, preferably 5 and 15 ng/ml, preferably 6, 7, 8, 9, 10, 11, 12, 13, 14, 14 ng/ml. Most preferably, the amount of activin is applied at 50 ng/ml, and then decreased to 25 ng/ml, then to 10 ng/ml over the time period. Preferably, the decrease in activin is distributed evenly over the time period. Thus, for example, where the time period for the first stage is 3 days, the first decrease will take place on day 2, and the second on day 3.

Wnt is also applied to the culture of undifferentiated stem cells. Wnt is a signalling protein, active in cell development and patterning in embryogenesis. “wnt” includes any protein or polypeptide having wnt biological activity, such as the ability to activate the same downstream signalling pathway. Included are isolated, purified and/or recombinant forms of wnt, functional derivatives and homologues, or fragments of wnt which retain the ability to promote cell development and patterning. Whilst any form of wnt may be used, wnt3a is preferred. Functional derivatives and homologues may share at least 60%, 65%, 70%, 75%, 80%, 85%, 90%, 91%, 92%, 93%, 94%, 95%, 96%, 97%, 98%, or 99% sequence identity with the wild type wnt sequence, for example as shown in FIG. 15, over the whole of the sequence or a fragment thereof. Fragments of wnt useful in the present invention may be at least 50%, 55%, 60%, 65%, 70%, 75%, 80%, 85%, 90%, 91%, 92%, 93%, 94%, 95%, 96%, 97%, 98%, or 99% the length of the full length wnt sequence, for example as shown in FIG. 15.

Wnt may be applied to the culture of undifferentiated stem cells, in combination with, simultaneously and/or sequentially to the application of activin. Preferably, wnt is applied for the period of time for a stem cell to differentiate into a mesendoderm cell. Preferably, this is simultaneous to the application of activin. Over this time period, multiple doses of wnt may be applied, preferably at daily intervals. Most preferably, daily successive doses of wnt are applied until a stem cell in the culture has differentiated into a mesendoderm cell. Whilst it is within the scope of the invention for the amount of wnt to be increased and/or decreased during its period of application, it is preferred that the amount of wnt applied to the culture remains the same until a stem cell in the culture has differentiated into a mesendoderm cell. Preferably, wnt is applied at between 15 and 45 ng/ml, preferably between 20 and 30 ng/ml, more preferably 21, 22, 23, 24, 25, 26, 27, 28 or 29 ng/ml.

FGF, or basis fibroblast growth factor, is a membrane bound protein. “FGF” includes any protein or polypeptide having FGF biological activity, such as the ability to bind heparin, bind FGF-receptors and/or heparin sulphate proteoglyrams, and,/or promote development and patterning. Included are isolated, purified and/or recombinant forms of FGF, other members of the FGF superfamily having the required activity, and functional derivatives or homologues, or fragments, of FGF having the aforementioned FGF activity. Functional derivatives and homologues may share at least 60%, 65%, 70%, 75%, 80%, 85%, 90%, 91%, 92%, 93%, 94%, 95%, 96%, 97%, 98%, or 99% sequence identity with the wild type FGF sequence, for example as shown in FIG. 15, over the whole of the sequence or a fragment thereof. Fragments of FGF useful in the present invention may be at least 50%, 55%, 60%, 65%, 70%, 75%, 80%, 85%, 90%, 91%, 92%, 93%, 94%, 95%, 96%, 97%, 98%, or 99% the length of the full length FGF sequence, for example as shown in FIG. 15.

Preferred FGFs for use in the invention are FGF1-10, more preferably FGF1-4, and most preferably FGF2.

FGF may be applied to the culture of undifferentiated stem cells, in combination with, simultaneously and/or sequentially to the application of activin and/or wnt. Preferably, FGF is applied for all or part of the period of time for a stem cell to differentiate into a mesendoderm cell. Preferably, for at least part of the time period taken for a stem cell to differentiate into a mesendoderm cell, the application of FGF is simultaneous to the application of activin and/or wnt. In a preferred embodiment, application of FGF may continue beyond the time period taken for a stem cell to differentiate into a mesendoderm cell, and may be applied during all or part of the time period for differentiation of a mesendoderm cell into a mesodermal lineage progenitor cell, and for all or part of the time period for a mesodermal lineage progenitor cell to differentiate into a chondro-, osteo-, and/or teno- progenitor cell. Over this time period, multiple doses of FGF may be applied, preferably at daily intervals. Most preferably, daily successive doses of FGF are applied Whilst it is within the scope of the invention for the amount of FGF to be increased and/or decreased during its period of application, it is preferred that the amount of FGF applied to the culture remains at the same level. Preferably, FGF is applied at between 4 and 30 ng/ml, preferably between 15 and 25 ng/ml, more preferably 16, 17, 16, 19, 20, 21, 22, 23, 24, or 25 ng/ml.

BMP, or Bone Morphogenic Protein, is a member of the TFGβ superfamily bound protein. It is regulated by binding proteins such as noggin and chordin, and involved in mesoderm formation. “BMP” includes such activity isolated, purified and/or recombinant forms of BMP, other members of the TGFβ superfamily having the required activity, functional derivatives and homologues, and fragments, of BMP having BMP activity. Functional derivatives and homologues may share at least 60%, 65%, 70%, 75%, 80%, 85%, 90%, 91%, 92%, 93%, 94%, 95%, 96%, 97%, 98%, or 99% sequence identity with the wild type BMP sequence, for example as shown in FIG. 15, over the whole of the sequence or a fragment thereof. Fragments of BMP useful in the present invention may be at least 50%, 55%, 60%, 65%, 70%, 75%, 80%, 85%, 90%, 91%, 92%, 93%, 94%, 95%, 96%, 97%, 98%, or 99% the length of the full length BMP sequence, for example as shown in FIG. 15.

BMP may be applied to the culture of undifferentiated stem cells, simultaneously and/or sequentially to the application of activin and/or Wnt. Preferably, BMP is applied for all or part of the period of time for a stem cell to differentiate into a mesendoderm cell. Preferably, for at least part of the time period taken for a stem cell to differentiate into a mesendoderm cell, the application of BMP is simultaneous to the application of one or more factors selected from the group selected from activin, wnt, and FGF. In a preferred embodiment, application of BMP may continue beyond the time period taken for a stem cell to differentiate into a mesendoderm cell, and may be applied during all or part of the time period for differentiation of a mesendoderm cell into a mesodermal lineage progenitor cell and for all or part of the time period for a mesoderm cell to differentiate into a chondro-, osteo-, and/or teno-progenitor cell. Over this time period, multiple doses of BMP may be applied, preferably at daily intervals. Most preferably, daily successive doses of BMP are applied. Whilst it is within the scope of the invention for the amount of BMP to be increased and/or decreased during its period of application, it is preferred that the amount of BMP applied to the culture remains at the same level. Preferably, BMP is applied at between 10 and 60 ng/ml, preferably between 20 and 50 ng/ml, preferably between 30 and 50 ng/ml, preferably between 35 and 45 ng/ml, more preferably 36, 37, 38, 39, 40, 41, 42, 43, 44, or 45 ng/ml.

An inhibitor of activin is any factor which reduces or prevents the activity of activin, preventing activation of receptor signalling by whatever means. Inhibitors of activin include follastatin, and any protein or polypeptide having such inhibitory activity. Included are isolated, purified and/or recombinant forms of such inhibitors, functional derivatives and homologues or fragments of such inhibitors. A preferred inhibitor of activin is follastatin, for example that having the sequence as shown in Shimasaki et al Proc. Natl. Acad. Sci. USA 85 (1988) 4221. Functional derivatives and homologues of may share at least 60%, 65%, 70%, 75%, 80%, 85%, 90%, 91%, 92%, 93%, 94%, 95%, 96%, 97%, 98%, or 99% sequence identity with the wild type inhibitor sequence, for example that of follistatin. The sequence of human follistatin is as provided in Shimasaki et al Proc. Natl. Acad. Sci. USA 85 (1988) 4221, over the whole of the sequence or a fragment thereof. Fragments of inhibitors useful in the present invention may be at least 50%, 55%, 60%, 65%, 70%, 75%, 80%, 85%, 90%, 91%, 92%, 93%, 94%, 95%, 96%, 97%, 98%, or 99% the length of the full length inhibitor sequence, for example follistatin as provided in Shimasaki et al Proc. Natl. Acad. Sci. USA 85 (1988) 4221.

An inhibitor of activin is preferably applied to the culture sequential to the application of activin, to prevent its further action within the culture. The application may be in combination with, simultaneous or sequential to the application of one or more factors selected from the group consisting of activin, wnt, FGF and BMP. Preferably, an inhibitor of activin is applied for all or part of the period of time for a mesendoderm cell to differentiate into a mesodermal lineage progenitor cell. In a preferred embodiment, application of an inhibitor of activin may continue beyond the time period taken for a mesendoderm cell to differentiate into a mesodermal lineage progenitor cell and may be applied during all or part of the time period for differentiation of a mesodermal lineage progenitor cell into a chondro- osteo- and/or teno-progenitor cell. Over this time period, multiple doses of an inhibitor of activin may be applied, preferably at daily intervals. Most preferably, daily successive doses of the inhibitor are applied. Whilst it is within the scope of the invention for the amount of inhibitor to be increased and/or decreased during its period of application, it is preferred that the amount applied to the culture remains at the same level. Preferably, the inhibitor of activin is applied at between 70 and 130 ng/ml, preferably between 80 and 120 ng/ml, more preferably between 90 and 110, most preferably 95, 96, 97, 98, 99, 100, 101, 102, 103, 104 or 105 ng/ml.

GDF, or Growth Differentiation factor 5, is a member of the BMP and TGFβ superfamily of proteins. It has a role in skeletal and joint formation. “GDF” includes any protein or polypeptide which modulates differentiation of mesodermal cells toward tissue formation, which activity characterises GDF. Included are isolated, purified and/or recombinant forms of GDF, other members of the TGFβ superfamily having the required activity, functional derivatives and homologues or fragments of GDF having said activity. Functional derivatives and homologues of may share at least 60%, 65%, 70%, 75%, 80%, 85%, 90%, 91%, 92%, 93%, 94%, 95%, 96%, 97%, 98%, or 99% sequence identity with the wild type GDF sequence, for example that of GDF as shown in FIG. 15, over the whole of the sequence or a fragment thereof. Fragments of inhibitors useful in the present invention may be at least 50%, 55%, 60%, 65%, 70%, 75%, 80%, 85%, 90%, 91%, 92%, 93%, 94%, 95%, 96%, 97%, 98%, or 99% the length of a full length GDF sequence, for example GDF as shown in FIG. 15.

GDF may be applied to the culture of mesodermal lineage progenitor cells, simultaneously and/or sequentially to the application of FGF, BMP and NT. Preferably, GDF is applied for all or part of the period of time for a stem cell to differentiate into a chondros- osteo- and/or teno-progenitor cell from a mesodermal lineage progenitor cell. Preferably, for at least part of the time period taken for a mesodermal lineage progenitor cell to differentiate into a chondro-, osteo- and/or teno progenitor cell, the application of GDF is simultaneous and/or subsequence to the application of one or more factors selected from the group selected from FGF, BMP and NT. Preferably, the application of GDF may also be subsequent the application of an inhibitor of activin. Over the specified time period, multiple doses of GDF may be applied, preferably at daily intervals. Most preferably, daily successive doses of GDF are applied. Whilst it is within the scope of the invention for the amount of GDF to be decreased, or maintained at the same level throughout the period of application, it is preferred that the amount of GDF applied is increased. More preferably, the dosage of GDF is doubled after part of the time period of application, most preferably after 2 days. Preferably, GDF is initially applied at between 5 and 40 ng/ml, preferably between 20 and 50 ng/ml preferably 15 and 25, preferably 16, 17, 18, 19, 20, 21, 22, 23, 24 or 25 ng/ml. Preferably, after 2 days, GDF is then applied at a level of between 20 and 50, between 35 and 45 ng/ml, more preferably 36, 37, 38, 39, 40, 41, 42, 43, 44, or 45 ng/ml.

NT, neurotrophin, which maintains or promotes proliferation of human cells includes NT and any protein or polypeptide included are proteins related to NT, or having sequence identity, homology and/or structural similarity to NT. Included are isolated, purified and/or recombinant forms of NT, other members of the NT family having the required activity, functional derivatives and homologues or fragments of NT having said activity. Functional derivatives and homologues of may share at least 60%, 65%, 70%, 75%, 80%, 85%, 90%, 91%, 92%, 93%, 94%, 95%, 96%, 97%, 98%, or 99% sequence identity with a wild type NT sequence, for example that of NT as shown in FIG. 15, over the whole of the sequence or a fragment thereof. Fragments of inhibitors useful in the present invention may be at least 50%, 55%, 60%, 65%, 70%, 75%, 80%, 85%, 90%, 91%, 92%, 93%, 94%, 95%, ₉₆%, 97%, 98%, or 99% the length of the full length NT sequence, for example NT as shown in FIG. 15

NT may be applied to the culture of mesendoderm and/or mesodermal lineage progenitor cells, simultaneously and/or sequentially to the application of FGF, BMP, an inhibitor of activin and GDF. It may also be applied subsequently to the application of activin and wnt. Preferably, NT is applied for all or part of the period of time for a mesendoderm cell to differentiate into a mesodermal lineage progenitor cell, and preferably, for at least part of the time period taken for a mesodermal lineage progenitor cell to differentiate into a chondro-, osteo- and/or teno-progenitor cell. Over the specified time period, multiple doses of NT may be applied, preferably at daily intervals. Most preferably, daily successive doses of NT are applied. Whilst it is within the scope of the invention for the amount of NT to be increased or decreased during the time period over which is applied to the culture of cells, preferably the level of NT being applied is maintained the same. Preferably, NT is applied at between 0 and 10 ng/ml, preferably 1 and 3 ng/ml, more preferably, 1.5, 1.6, 1.7, 1.8, 1.9, 2, 2.1, 2.2, 2.3, 2.4, or 2.5 ng/ml.

The differentiation of a stem cell according to the invention can be observed using markers displayed by the cells, which change as differentiation takes place. During, or at the end of the first stage of the method, the cells will have wholly or partly adopted a mesendoderm phenotype, which may be recognised the presence of one or more markers selected from the group consisting of E-cadherin, OCT4, NANOG, GSC, and BRA. Preferably, E-cadherin shows a 1-3 fold increase, preferably a 1.5-2 fold increase, most preferably a 1.6, 1.7,. 1.8, 1.9 or 2 fold increase compared to that of an undifferentiated stem cell. Preferably, GSC shows a 2-4 fold increase, preferably a 3-4 fold increase, most preferably a 3.1, 3.2, 3.3, 3.4, 3.5, 3.6, 3.7, 3.8 or 3.9 fold increase compared to that of an undifferentiated stem cell. Similarly, BRA showed a 500-1000 fold increase, preferably a 600-900, preferably 650, 700, 750 or 800 fold increase compared to an undifferentiated stem cell. Although during the first stage of the method of the invention an increase in cell death may be observed, there may be a concurrent increase in cell division, thereby maintaining cell numbers.

During or by the end of the second stage in which a mesendoderm cell differentiates into a mesodermal lineage progenitor cell, there may be observed an increase in confluency of the cells. Of the cellular markers, there may be observed a decrease or loss of OCT4 and/or SOX2 compared to those of stem cells, preferably a decrease of between 0 and 2% for OCT4, and 0 and 5% for SOX2. E-cadherin and GSC may also be reduced, preferably by between 2 and 10%, more preferably 6% and 8% respectively. GATA4, FOXA2 and SOX17 will also show reductions in expression levels. In addition, an increase in the markers MIXL1, CXCR4 and SOX0 may be observed.

During or by the end of the optional final stage of differentiation from a mesodermal lineage progenitor cell to a chandro, osteo and/or teno progenitor cell, an increase in expression of GDF5, PDGFRB, COL2AI, SOX9 and SOX6 is observed. Loss of expression of OCT4 NANOG, SOX2 E-cadherin is also observed, indicating a loss of pluripotency. Intermediate markers GSC, MIXL1 and BRA are lost and FLK1 and CXCR4 show reduced expression, of 0 to 1%, and 20 to 30%, respectively. The presence of sGAG is also observed for the first time in these differentiated cells.

The cells during or at the end of this final stage are smaller than the undifferentiated stem cell, and are flatter, show lower cell density, and a rounded, chondro-, osteo- and/or teno-cyte-like morphology and show increased dissociation from a substrate on which the cells are grown.

Preferably, at least 40%, 45%, 50%, 55%, 60%, 65% 70%, 75%, 80%, 85%, 90%, 96%, 98%, 99%, or 100% of the cells in the culture resulting from the final stage of the method of the invention express SOX9. Thus, the present invention provides a method for producing a cell population from a population of undifferentiated stem cell, in which at least 40%, 45%, 50%, 55%, 60%, or 65% 70%, 75%, 80%, 85%, 90%, 96%, 98%, 99%, or 100% of the cells in the culture express SOX9. This factor is chondrocyte associated transcription factor, and indicative of the differentiation of the cells toward a chondrocyte lineage. Preferably at least 20%, 25%, 30% or 35% of the cells express CD44.

The above mentioned markers may be monitored using any suitable techniques known and available to persons skilled in the art. Preferably, gene expression analysis may be used. Other suitable methods include transcript analysis, QPCR, protein, staining and FACS.

Preferably, the present invention does not require the use of feeder cells to support the growth of the stem cells, i.e. it is feeder-cell free. Generally speaking, where feeder cells are not used it may be advantageous to grow the cells on a compatible culture surface, preferably using a growth medium that provides some of the influences which would have been provided by the feeder cells. Particularly suitable substrates on which the cells may be grown are extracellular matrix components. Commercial preparations based on extracellular matrix components are known and available in the art, including for example Matrigel (Becton Dickenson). Depending upon the cell type being proliferated, other suitable extracellular matrix components and component mixtures may be used. These include laminin, fibronectin, proteoglycans, entactin, heparan sulfate, gelatin and other similar components such as sulphated GAG's and collagens, which will be known to persons skilled in the art. The components may be used alone or in combination. One or more different substrates or combinations of substrates may be used sequentially at different stages of growth and differentiation of the cells. For example, in a preferred embodiment, the stem cells may be initially grown on a fibronectin substrate, and later transferred to a different substrate or combination of substrates. Preferably, the cells are grown on fibronectin for the first stage and all or part of the second stage at least. During, or at the end of the second stage, the cells may be transferred to a second substrate, for example gelatin.

Preferably, the medium in which the cells are differentiated is serum free. Most preferably, the cells are grown in a base medium, which may comprise one or more of the factors listed in Table 1. Other suitable base cultures will be known and available to persons skilled in the art. Preferably, any base culture used will be suitable for enhancing the survival of the cells.

The cells will be plated onto a substrate in a suitable distribution and preferably in the presence of a medium which promotes growth and enhances survival of the cells. The seeding density of the cells on the substrate will preferably be at least 10e4 to 2×10e 6 cells/ml. The cells may be dispersed into a single cell suspension, or may be kept together in clusters, for example about 10-2000 cells in size. The clusters of cells are then plated onto a substrate.

Preferably, the method of the invention is performed in vitro.

The present invention also provides a culture of cells, as described herein. Such a culture may additionally comprise base medium, and one or more factors selected from the group consisting of activin, Wnt, BMP, FGF, an inhibitor of activin, GDF, and NT and optionally nodal BMP-2 and BMP-7. The present invention also provides a cell produced by the method of the invention. Preferably the cell is selected from the group consisting of L- a mesendoderm cell, a mesodermal lineage progenitor cell, a mesoderm cell, a chondro-, osteo-, and/or teno-progenitor cell, and a chondro-, osteo- and/or teno-cyte cell. Preferably, a cell produced by a method of the invention will exhibit reduced levels of Coll II, PDGFRB and/or sulphonated GAGs compared with a native cell of the same type. Such comparisons of cell markers can be made using known procedures available in the art, such as qPCR and protein straining. A native cell for use in such a comparison is one of the same type, and which has differentiated in vivo.

A cell or a culture of cells of the present invention may be used to treat bone cartilage and/or tendon-based defects. Thus, there is provided a cell or a cell culture as described herein, preferably produced by a method of the invention, for use in the treatment of such defects.

Herein, cartilage based defects may be the result of a traumatism, a birth defect or a disease such as osteoarthritis, and may be present in any joint of the body. Said defects may include “wear” and “loss” of cartilage.

Also provided is a therapeutic composition, comprising a cell or a culture of cells according to the invention.

The present invention also provides a matrix comprising cells of the invention, and which may be used in the production of a bone, tendon and/or cartilage-based product for treatment of such defects in the subject. The matrix is preferably seeded with stem cells, mesendoderm cells, mesoderma cells, mesodermal lineage progenitor cells, chondro- osteo-, and/or teno-progenitor cells, and/or chondro-, osteo-, and/or teno-cyte cells. In addition, the matrix may comprise one or more factors selected from the group consisting of activin, wnt, FGF, BNP, GDF and NT and an inhibitor of activin. The matrix may additionally comprise components of the extracellular matrix.

Preferably, the matrix is shaped to conform to its use as a part or all of a cartilage, bone or tendon which is to be repaired, reconstructed, augmented or replaced.

The matrix is preferably formed of a biocompatible material, which is any substance not having toxic or injurious effects on biological function. The matrix is preferably porous to allow for cell deposition both on and in the pores of the matrix. The shaped matrix may then contacted, preferably sequentially, with at least one cell population supplied to the matrix to seed the cell population on and/or into the matrix. The seeded matrix may be implanted in the body of the recipient where the separate, laminarily organized cell populations facilitate the formation of neo-organs or tissue structures. In some cases, it may be preferable for the matrix to be biodegradable. Biodegradable refers to material that can be absorbed or degraded in a patient's body. Representative materials for forming the biodegradable structure include natural or synthetic polymers, such as, for example, collagen, poly(alpha esters) such as poly(lactate acid), poly(glycolic acid), polyorthoesters andpolyanhydrides and their copolymers, which degraded by hydrolysis at a controlled rate and are reabsorbed. These materials provide the maximum control of degradability, manageability, size and configuration. Preferred biodegradable polymer material include polyglycolic acid and polyglactin, developed as absorbable synthetic suture material. Polyglycolic acid and polyglactin fibers may be used as supplied by the manufacturer. Other biodegradable materials include cellulose ether, cellulose,cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone,polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene,polythioether, polytriazole, polyurethane, polyvinyl, polyvinylidene fluoride, regenerated cellulose, silicone, urea-formaldehyde, or copolymers or physical blends of these materials. The material may be impregnated with suitable antimicrobial agentsand may be colored by a color additive to improve visibility and to aid in surgical procedures. Non-biodegradable materials include Teflon, polystyrene, polyacrilate or polyvinyl, or a protein hydrogel, or carbohydrate hydrogel.

The matrix can comprise or be coated with a second material such as gelatin to increase the bonding of the cells to the polymer. It may be flexible or rigid. A sponge-type structure can also be used, or macroporous microcarriers.

Finally, the present invention also provides a kit of parts, comprising in separate containers one or more factors selected from the group consisting of activin, Wnt, BMP, FGF, an inhibitor of activin, GDF, and NT. The kit may also comprise a base medium, a starting culture of cells, a substrate, a gel and/or scaffold, or feeder cells, and instructions for use.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### EXAMPLES

### Directed Differentiation of hES Cells to Chondrocytes

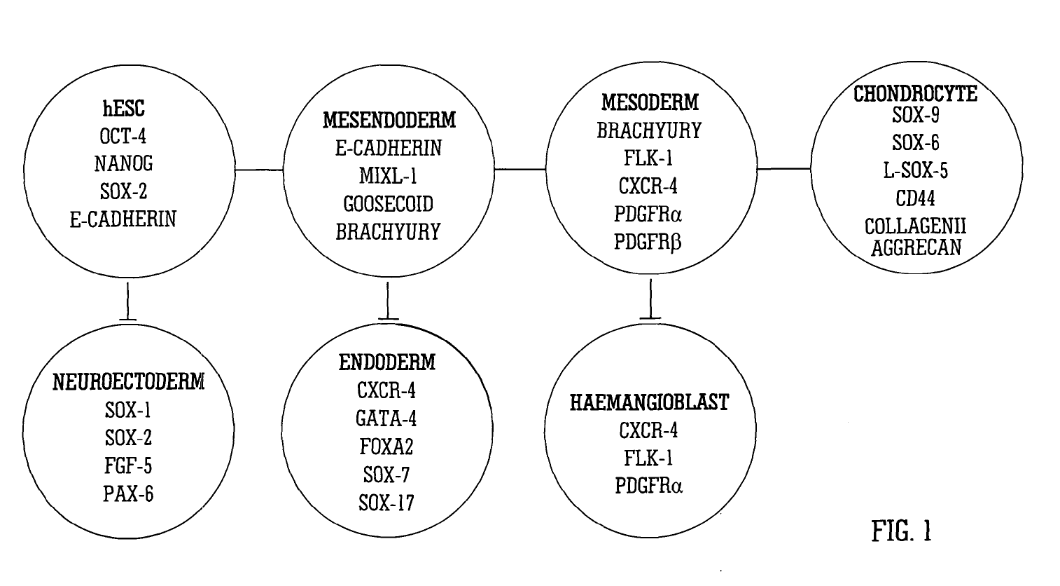

With the aim of generating chondrocytes, the initial differentiation protocol was based on the known developmental progression of cells to bi-potent mesendoderm and then to mesoderm. This was developed using the HUES1 human embryonic stem (hES) cell line into a 3-step directed differentiation protocol (FIG. 1, Table 1).

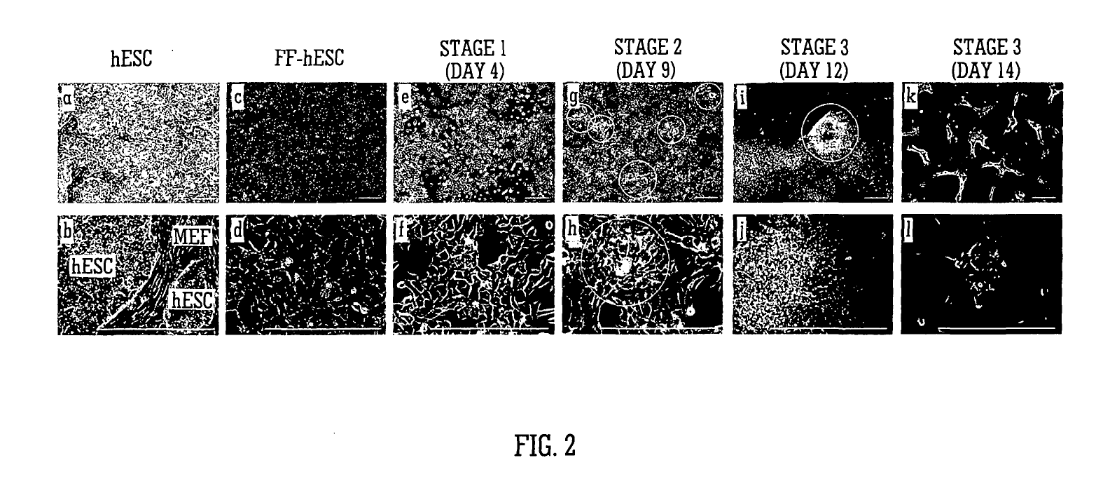

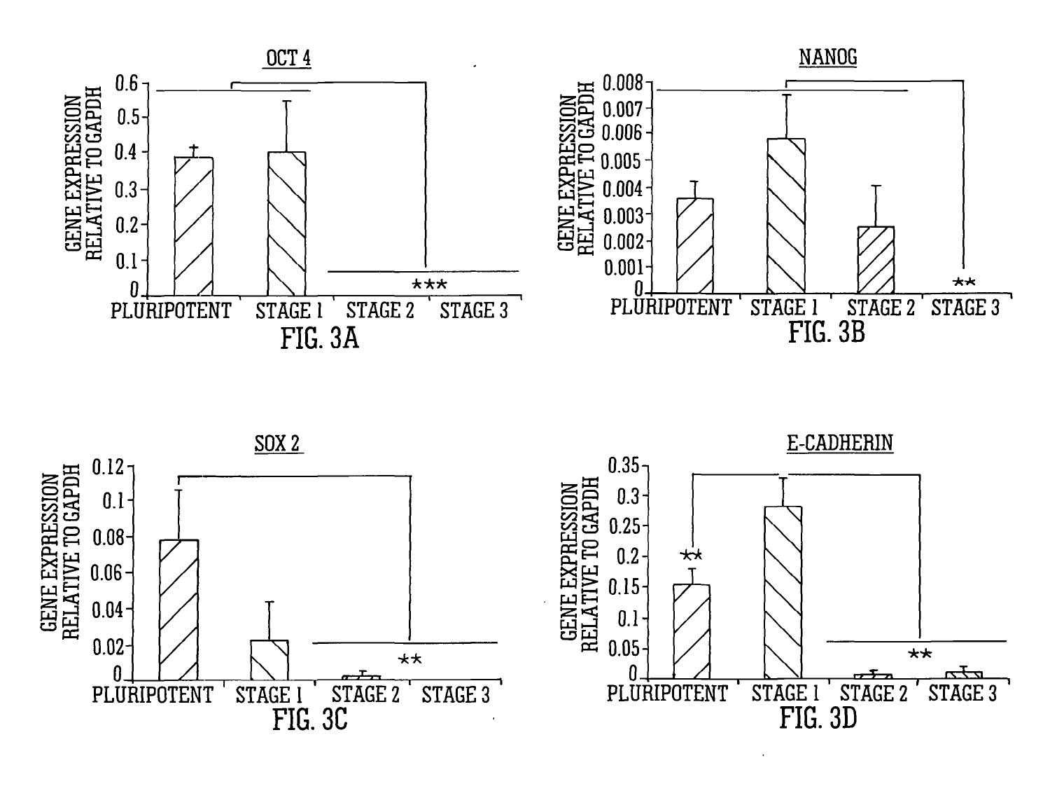

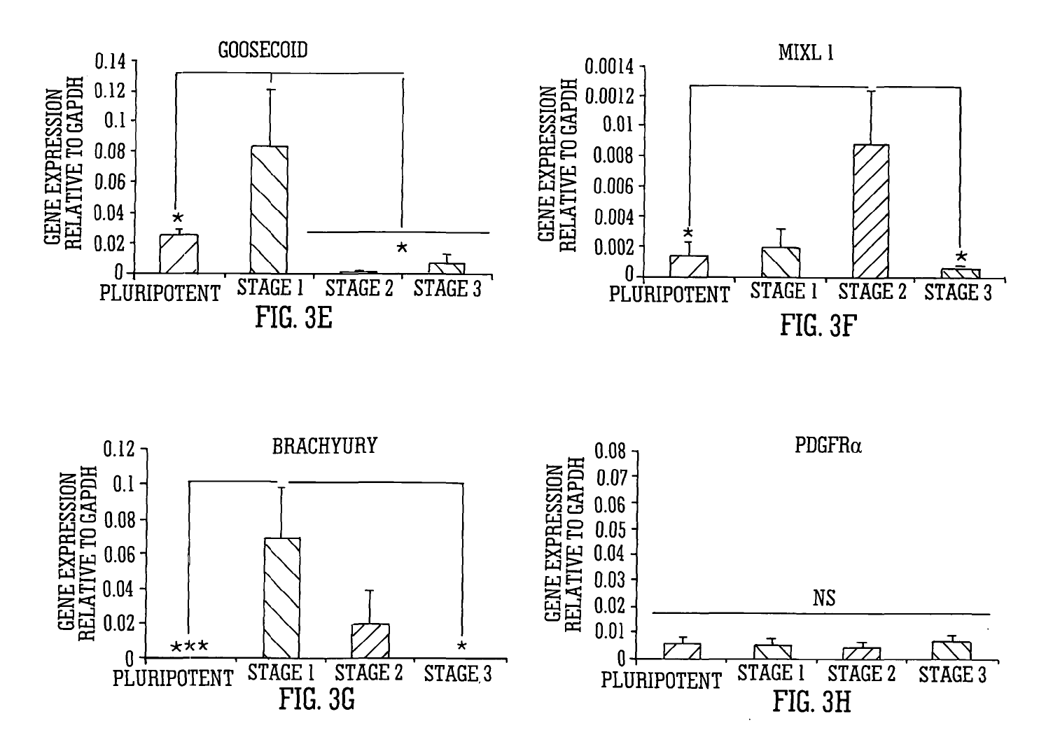

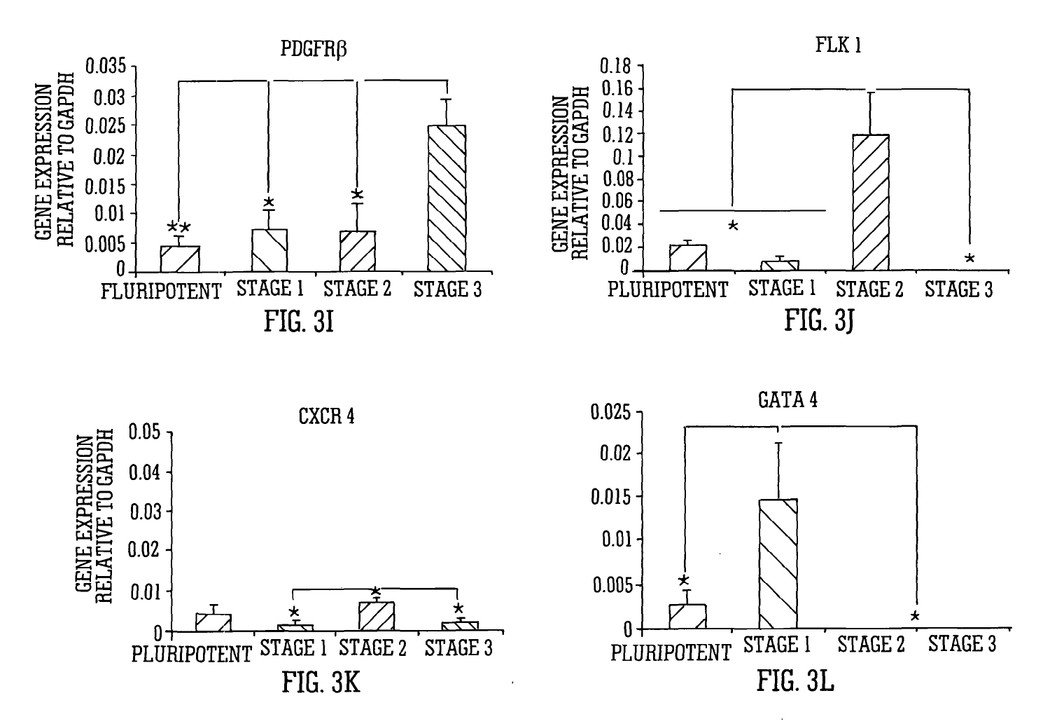

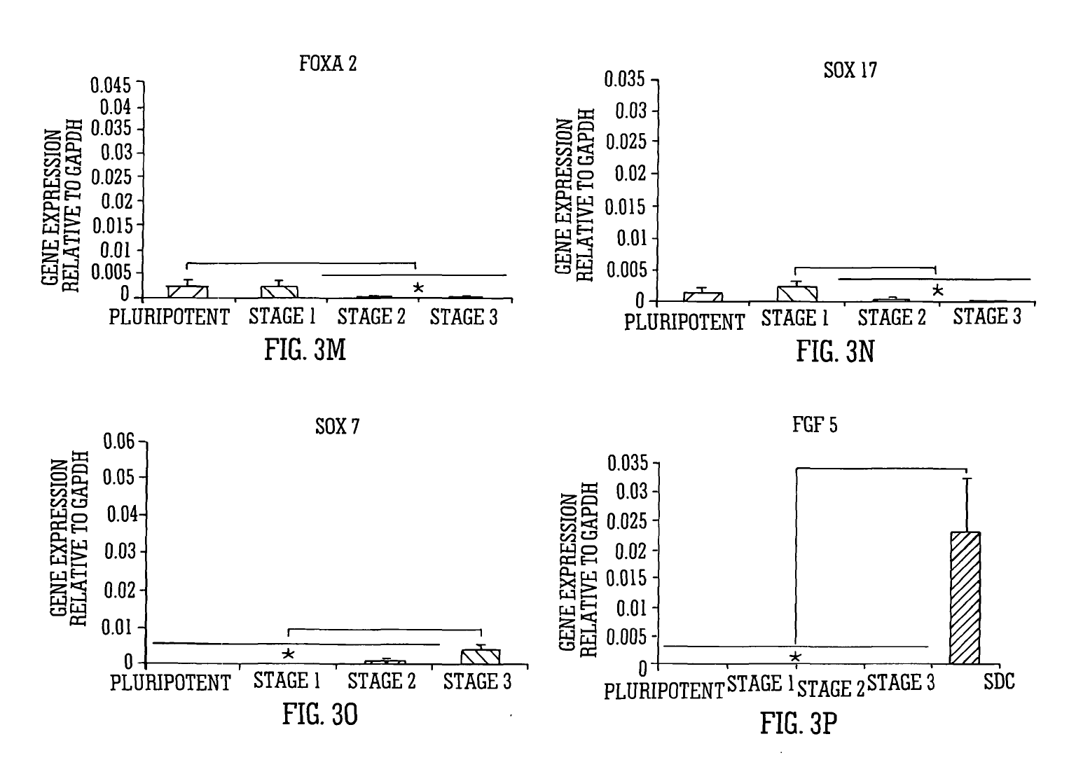

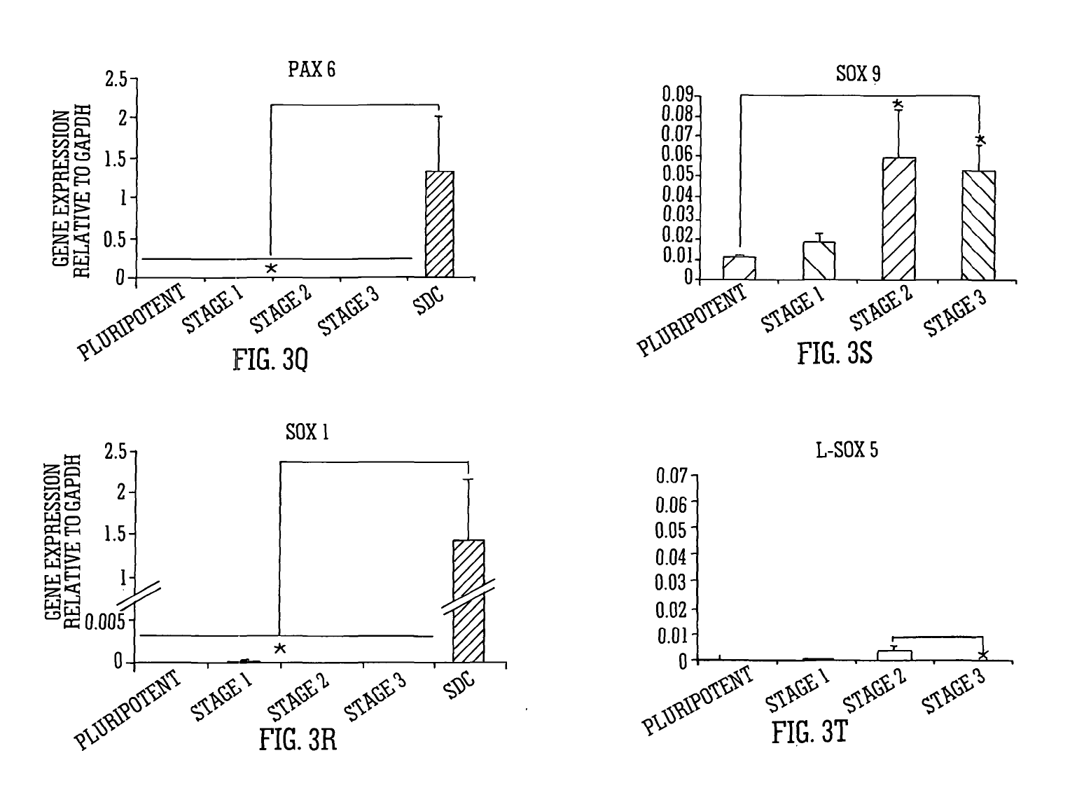

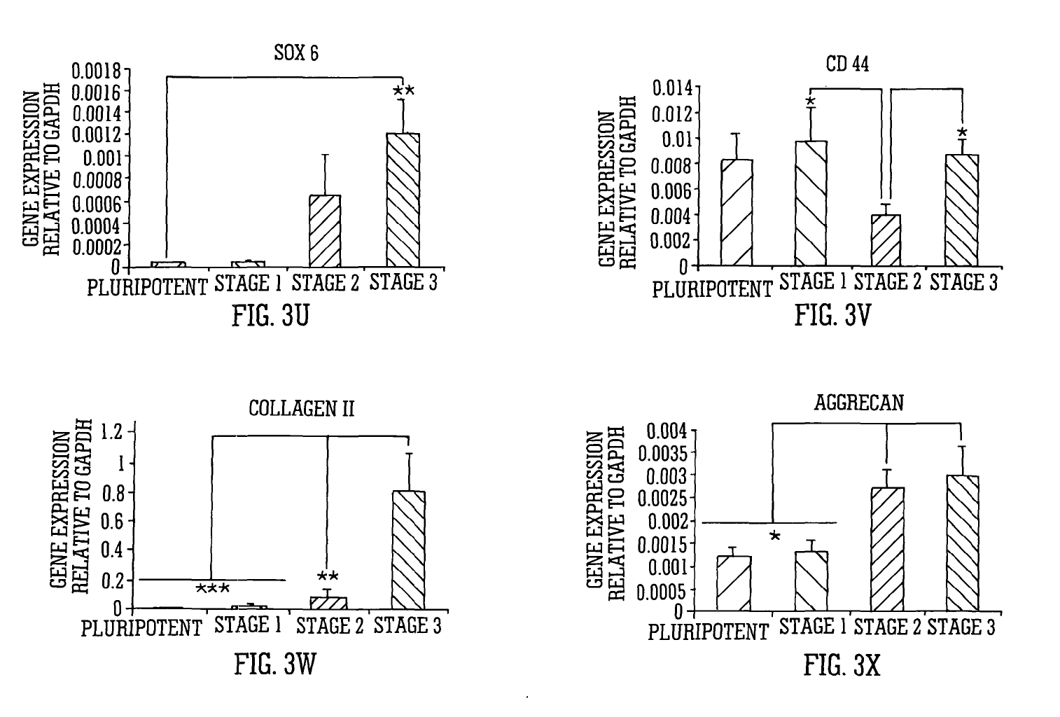

Importantly, starting hES cells were maintained as feeder-free, serum-free cultures (Baxter, M. A. et al. Stem Cell Res 3, 28-38 (2009)) (FIGS. 2a-d). The protocol began with the hES cell culture approximately 80% confluent and initial differentiation used a defined serum-free basal medium. Refinement of pilot protocols involved testing different growth factor combinations and concentrations and varying the times of their addition and their duration (Supplementary Methods online). Cell progeny were assayed throughout the culture for expression of pluripotency-associated genes (FIGS. 3a-d) and mesendodermal (FIGS. 3e-f), mesodermal (FIGS. 3g-k), endodermal (FIGS. 3l-o), neurectodermal (Figs. p-r) and chondrocyte (FIGS. 3s-x) cell lineage markers. This enabled refinement of the protocol to achieve more efficient differentiation. The cells in the protocol were compared with those undergoing spontaneous undirected differentiation when cultured over an equivalent time in the presence of serum.

### Development of the Protocol

Stage 1 of the protocol (days 1-3) was aimed at driving the differentiation of pluripotent hES cells to bi-potent mesendoderm population based upon previous results with RPMI-base medium containing wnt3a and activin-A (D'Amour, K. A. et al. Nature Biotechnol 24, 1392-1401 (2006)). In pilot experiments we were unable to maintain hES cell cultures under serum-free, feeder-free conditions using RPMI medium. Therefore an alternative base medium (see Methods) was developed which was able to support hES cell viability. In this medium the initial regime of wnt3a and activin-A described by D'Amour et al. (2006) was partly successful, but resulted in high expression of genes associated with both extra-embryonic and definitive endoderm. The protocol was therefore progressively modified reducing the initial activin-A to 50 ng/ml and lowering it to 25 ng/ml at day 2 and 10 ng/ml at day 3. The application of wnt3a (25 ng/ml) was also extended to day 3 with FGF2 (20 ng/ml) from day 2 onwards and BMP4 (40 ng/ml) on day 3. During Stage 1 the number of cells within the culture approximately doubled (Supplementary FIG. 1 online). There was no apparent change in cell morphology during Stage 1 (FIG. 2e, f) with cells retaining the large nuclei with prominent nucleoli of hES cells. The pluripotency genes OCT4 and NANOG continued to be expressed at levels comparable to hES cell cultures (FIGS. 3a, b). Whilst SOX2 appeared to decrease, the difference in expression between pluripotent hES cell cultures and Stage 1 cultures was not statistically significant (FIG. 3c). However, there was evidence of differentiation towards a bi-potent mesendodermal population by the end of Stage 1. The cell adhesion molecule E-cadherin is expressed by pluripotent hES cells (Eastham, A. M. et al. Cancer Res 67, 11254-11262 (2007)) and is also associated with a mesendoderm phenotype (Tada, S. et al. Development 132, 4363-4374 (2005)) and its expression was accordingly upregulated by 1.8-fold (P<0.01) in Stage 1 (FIG. 3d). Similarly, GSC expression also increased 3.3-fold (P<0.05) (FIG. 3e) and BRA, a gene expressed by early mesoderm (Kispert, A. Genes Dev 8, 2137-2150 (1994)), but which has also been shown to be expressed by murine ES cells with potential for both mesoderm and endoderm (Kubo, A. et al. Development 131, 1651-1662 (2004)), rapidly increased 800-fold (P<0.001) (FIG. 3g). Immunofluorescence at Stagel showed that BRACHYURY and GOOSECOID were expressed by over 95% of the cell population. Consistent with a pluripotent population, flow cytometry revealed E-CADHERIN on 95% of hES cells and the proportion was unchanged through Stage 1. Immunolocalization was consistent with the flow cytometry data and showed that E-CADHERIN was expressed more strongly in some regions of the cell culture (Supplementary FIG. 2 online). At Stage 1, a 5-fold increase in GATA4 was detected P<0.05) (FIG. 3l), which is typically associated with cell populations committed to definitive endoderm. These data together suggest that Stage 1 yielded a cell population enriched for bi-potent mesendoderm,

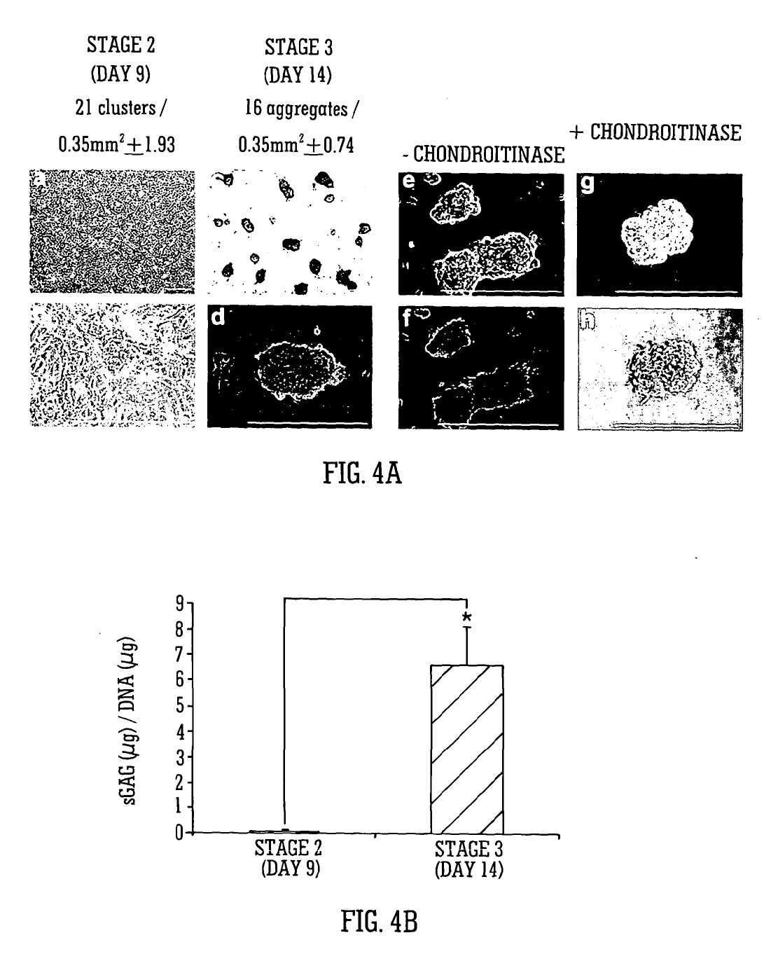

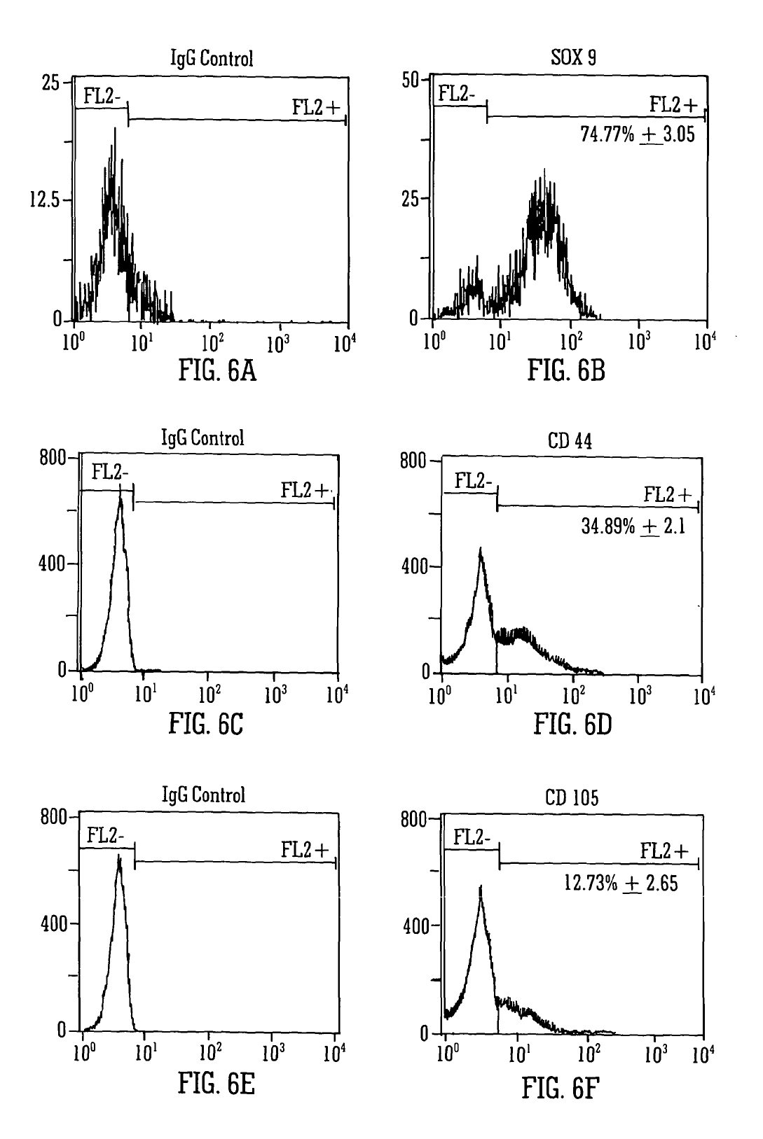

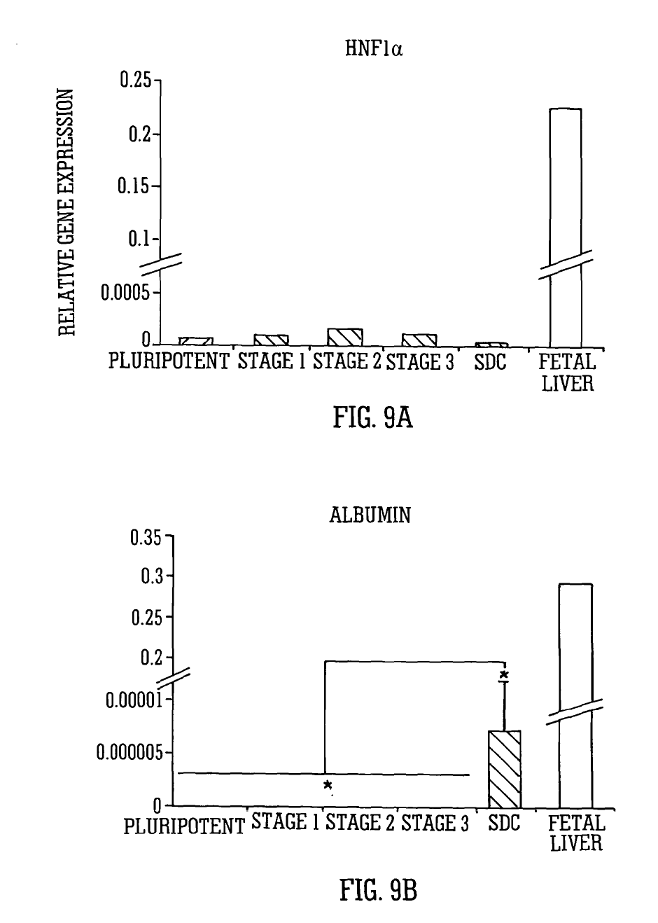

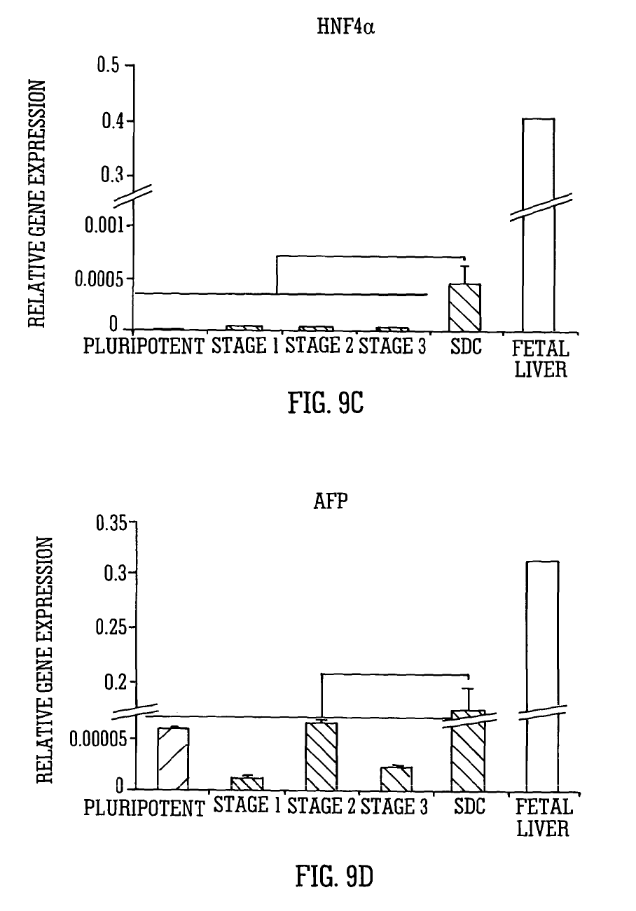

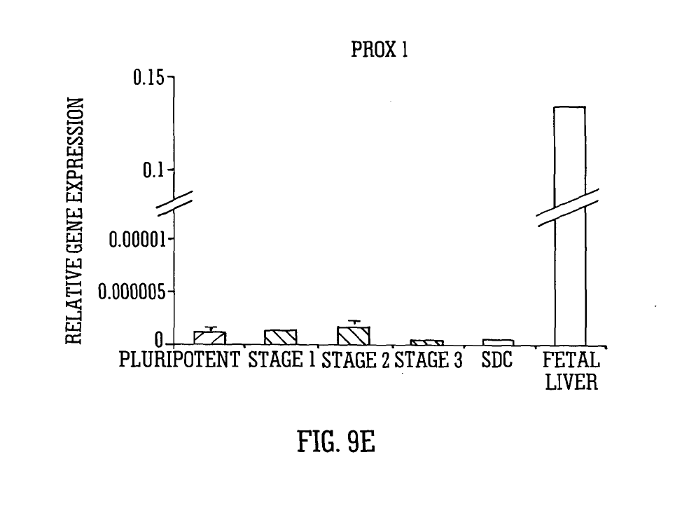

During Stage 2 of the protocol, (day 4-8) BMP4 and FGF2 supplementation were continued and wnt3a and activin-A were removed and the expression of genes involved in the specification of endoderm were reduced further by including follistatin (100 ng/ml). In Stage 2 Neurotrophin-4 (NT4) (2ng/ml) was added which promoted cell survival (Pyle, A. D., Nature Biotechnol 24, 344-350 (2006)). During this stage cultures were expanded by two passages, reflecting a high proliferation rate (FIG. 2g) (FIG. 6). At the end of Stage 2 (FIG. 2g, h) the cells were 95% confluent and contained occasional phase-bright cell clusters ˜φ □m in diameter (circled) which occurred at a frequency of 21 clusters/0.35 mm²±SEM 1.93 (N=4) and these stained with safranin O, indicative of the accumulation of sGAG (FIG. 4a, b). Gene expression analysis showed loss of OCT4 and SOX2 expression to 0.1% (P<0.001) and 3.3% (P<0.01) that of pluripotent hES cells respectively. In addition ECAD expression was reduced to 6% (P<0.01) and GSC to 8% (P<0.05) of the values recorded at Stage 1. MIXL1 expression, often used to identify mesendodermal cells, was up-regulated later than predicted, increasing 6.3 fold by the end of Stage 2, compared with pluripotent hES cells (P<0.05) (FIG. 3f). However, there was also evidence of differentiation to an intermediate mesoderm cell population at the end of Stage 2; FLK1, expressed by multipotent mesoderm (Ema, M. Blood 107, 111-117 (2006)). increased 5.5 fold (P<0.05) between Stages 1 and 2 (FIG. 3j) and CXCR4, used to characterize a mesodermal population that was destined to form hemangioblastic cell lineages (Kubo, A. et al. Development 131, 1651-1662 (2004)) was also up-regulated by 1.5-fold (P<0.05) over the same period (FIG. 3k). Significantly, SOX9, which is expressed in all chondrogenic cells (kiyama, H et al Genes Dev 16, 2813-2828 (2002); Bi, W., Nature Genet 22, 85-89 (1999)), but also in early development in other lineages, was upregulated 5-fold (P<0.05) at the end of Stage 2 (FIG. 3s) and there was much reduced expression of genes associated with cell lineages derived from definitive endoderm. GATA4, which had been high at Stage 1 declined to just 0.5% of that level by Stage 2 (P<0.05) (FIG. 3l) and the expression of FOXA2 and SOX17 was significantly reduced compared to pluripotent cultures (FIGS. 3m, n). To check for cells destined for other lineages, the transcript levels for genes expressed much later in endoderm differentiation were analysed and there was no detectable expression of HNF1a, HNF4a, PROX1, ALB or AFP at any stage of our directed differentiation protocol (FIG. 9) confirming that the detected increase in SOX9 expression was not associated with differentiation towards primitive hepatocyte lineages.

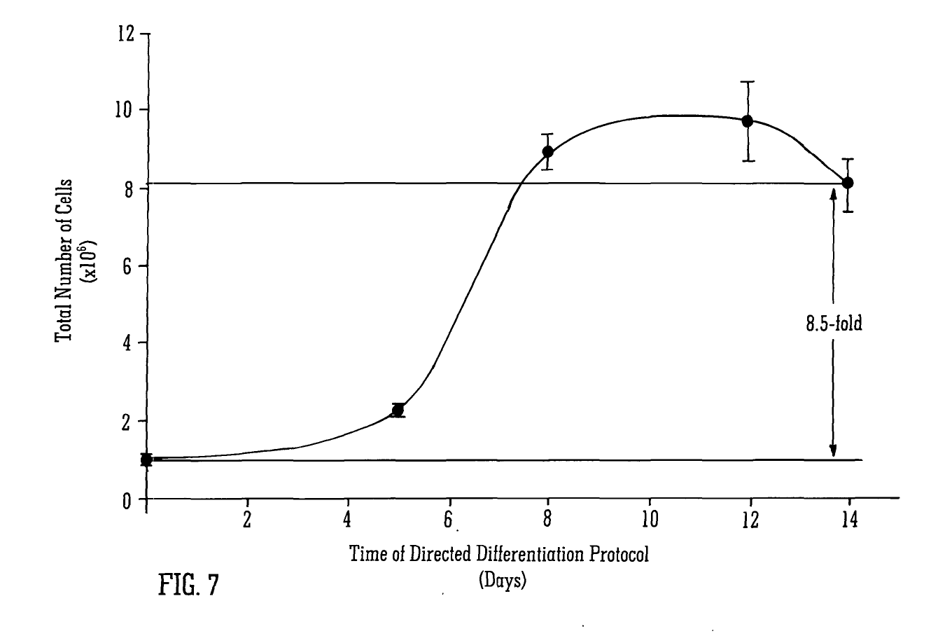

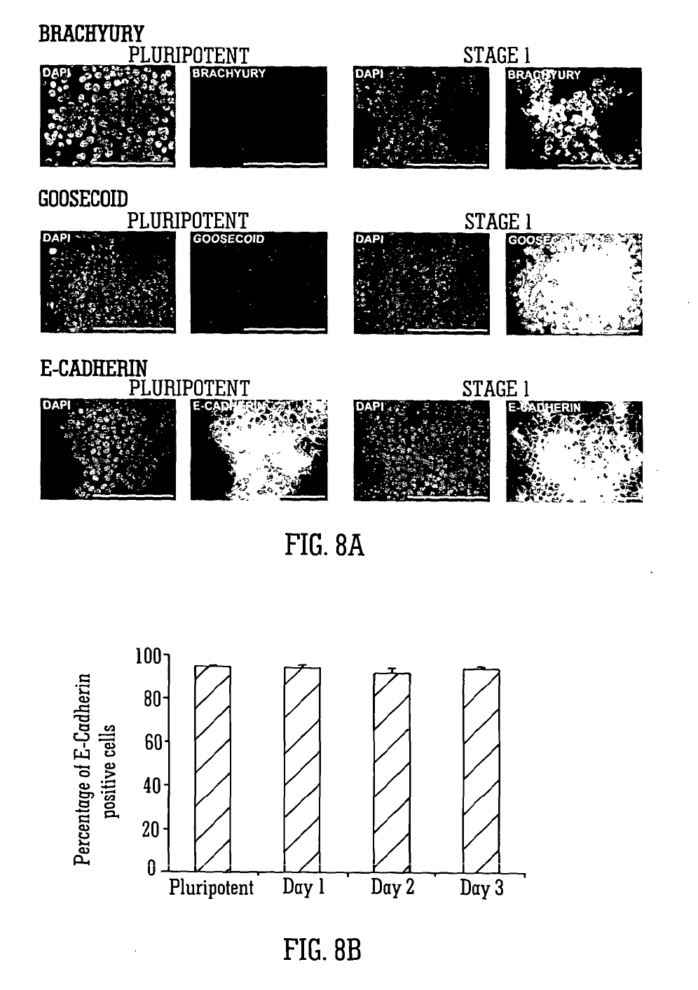

Stage 3 of the protocol (day 9-14) included a graded switch from BMP4 to GDF5, which was increased to 40 ng/ml by day 11. By day 12 (prior to passage) the cell clusters formed during Stage 2 had increased in size (circled) (FIGS. 2i, j) and the flatter cells between the cell clusters, were less firmly attached to the culture dish. At the end of Stage 3 the cell density was lower than in earlier stages (FIGS. 2k, l) though there was a 8.5-fold (P<0.05) increase in total cell numbers over the course of the directed differentiation protocol (FIG. 7). The flatter cells were no longer present leaving independent aggregates of cells with a rounded chondrocyte-like morphology. The formation of these cell aggregates appeared comparable to the initial stages of chondrogenesis, and these cell aggregates were larger and occurred at a lower frequency than the cell clusters at Stage 2 (16 aggregates/0.35 mm²±SEM 0.74 (N=4)). They also stained more strongly for safranin O (FIG. 4c, d) and the staining was sensitive to chondroitinase ABC removal of chondroitin sulphate, which confirmed that the cell aggregates accumulated sGAG within the matrix (FIG. 3e-h). The accumulation of sGAG was quantified (FIG. 3i) and cultures showed a 78-fold increase in sGAG production per cell between Stage 2 and Stage 3 cultures (P<0.05).

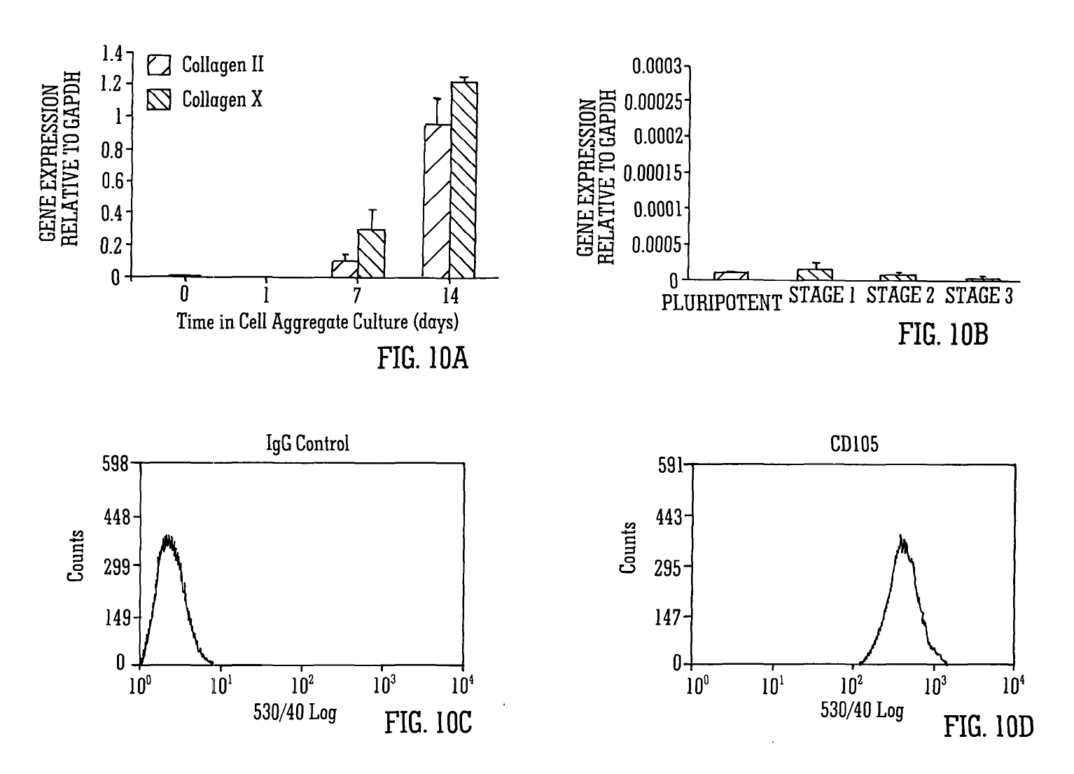

These did not express OCT4, NANOG, SOX2 and ECAD, indicating a complete loss of pluripotency and there was no detectable expression of genes associated with intermediate cell populations including GSC, MIXL1 and BRA. Expression of FLK1 declined to 0.005% (P<0.05) of the values observed in Stage 2, suggesting that the protocol directed differentiation away from a hemangioblastic population. Surprisingly, there was no change in expression throughout the protocol of PDGFRA, which is reportedly associated with mesodermal cells (Tada et al Development 132, 4363-4374 (2005)) Takenaga, M., et al J Cell Science 120, 2078-2090 (2007)) (FIG. 3h), though its homologue, PDGFRB (Tada et al Development 132, 4363-4374 (2005), Betsholtz, C. et al Bioessays 23, 494-507 (2001)) was increased in expression 5.5 fold at the end of Stage 3 (P<0.01) (FIG. 3i). The presence of chondroblastic cells at the end of Stage 3 was assessed by expression of SOX9, L-SOX5, SOX6, CD44, COL2A1 and AGCAN (FIGS. 3s-x). SOX9 expression was comparable to that observed in Stage 2 and there was also 3.7 fold up-regulation of its transcription co-factor, SOX6, (P<0.01). Whilst some modest up-regulation of L-SOX5, was recorded at the end of Stage 2, it was inconsistent and expression fell by half at the end of Stage 3 (P<0.05). Pluripotent hES cells and cells at Stage 1 expressed similar amounts of CD44, possibly reflecting the role of hyaluronan in the maintenance of pluripotency and early cell fate decisions (Choudhary, M. et al. Stem Cells 25, 3045-3057 (2007)). Expression was lost during the intermediate stages of differentiation, before returning to the level observed in pluripotent hES cell cultures. COL2A1, a downstream gene target of SOX9 (Bell, D. M. et al. Nature Genet 16, 174-178 (1997) Lefebvre, V., Mol Cellular Biol 17, 2336-2346 (1997)) and key cartilage matrix component showed a 370-fold increase between pluripotent hES cells and the end of Stage 3 (P<0.01). AGCN, another component of cartilage ECM was also upregulated 2.5-fold between pluripotent hES cells and the end of Stage 3 (P<0.05). COL10A1, expressed by hypertrophic chondrocytes undergoing terminal differentiation, was not detected in our directed differentiation protocol in contrast to CD105-positive MSCs undergoing chondrogenic differentiation (FIG. 10).

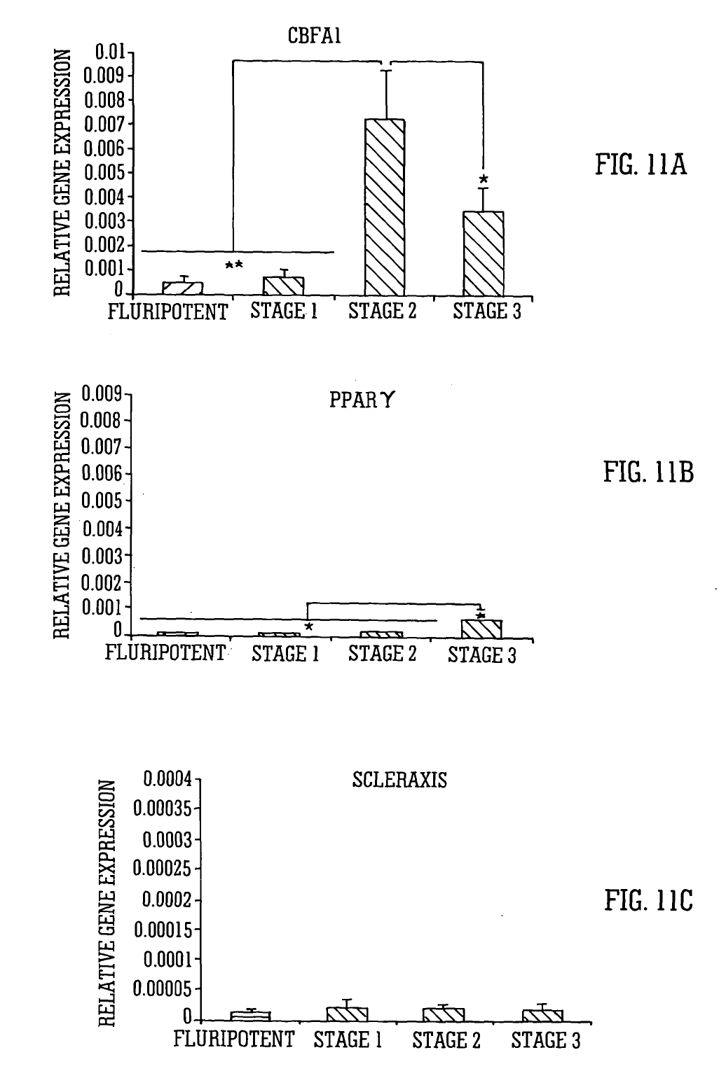

To further understand the developmental phenotype of the cells generated by this directed differentiation protocol we also investigated the regulation of genes expressed by related mesodermal cell lineages (FIG. 11). CBFA1, a transcription factor expressed during osteoblast differentiation (Ducy, P. Dev Dyn 219, 461-471 (2000)) was shown to be upregulated 10-fold between Stage 1 and Stage 2 (P<0.01) before decreasing 50% at the end of Stage 3 (P<0.05). PPAR□, a transcription factor that regulates adipogenesis (Rosen, E. D. Prostaglandins, leukotrienes, and essential fatty acids 73, 31-34 (2005)) was expressed at a low level throughout directed differentiation and was upregulated by 6-fold between Stage 2 and 3 (P<0.05). SCLERAXIS, a transcription factor expressed highly during tenogenic differentiation (Schweitzer, R. et al. Development 128, 3855-3866 (2001)) showed low expression throughout the protocol. These data suggest that our directed differentiation protocol is specific for differentiation toward chondrocytes.

### Immunolocalization of Cells at Stage 3

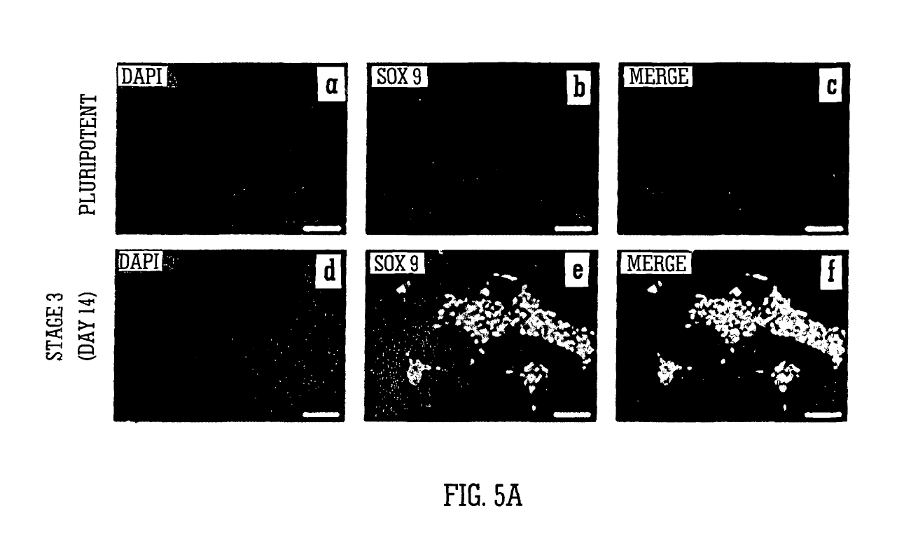

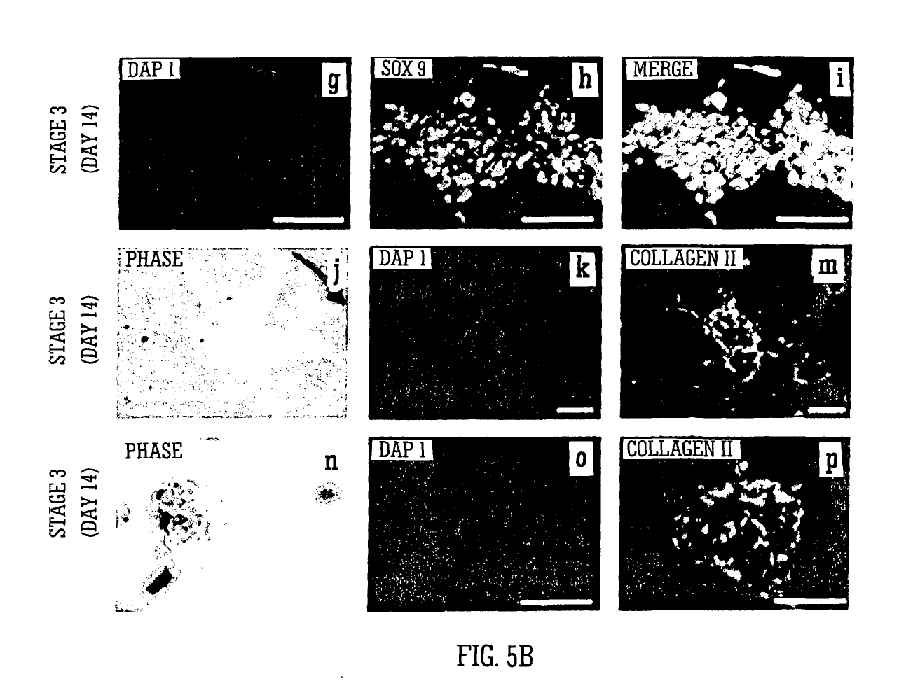

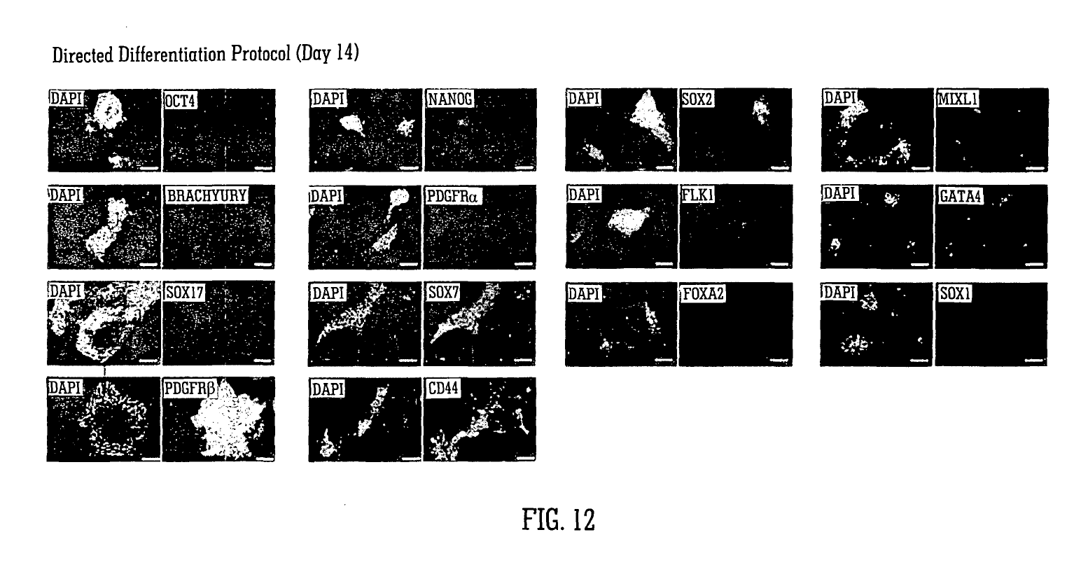

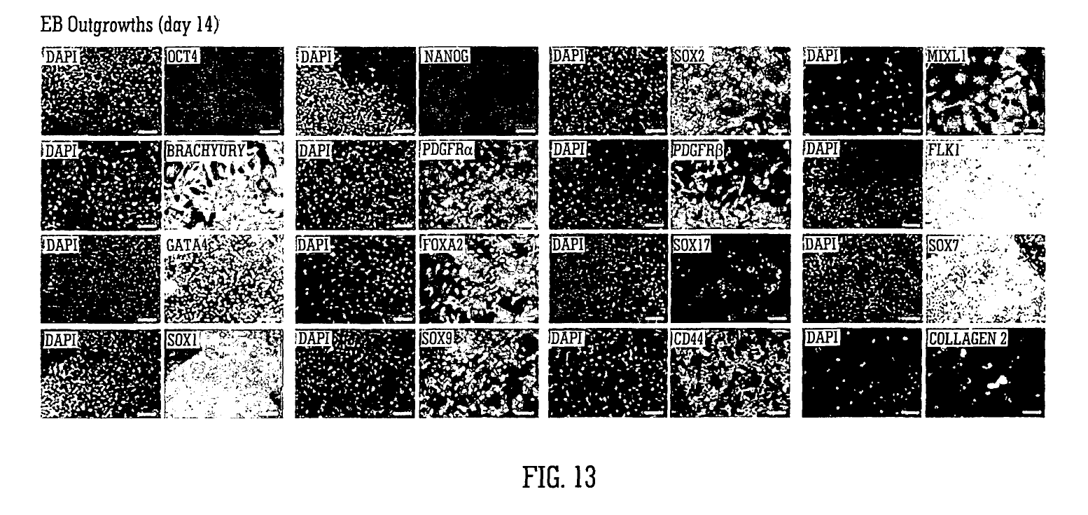

Stage 3 cultures were analyzed by immunolocalization for expression of differentiation markers (FIG. 5 and FIG. 12) and compared to spontaneously differentiating cultures (FIG. 13) from embryoid bodies. The latter contained heterogeneous cell populations which expressed proteins associated with all three germ layers as well as developmental intermediate populations. In contrast, Stage 3 cultures were more homogeneous with little non-target marker expression (FIG. 12). Immunostaining for OCT4 and NANOG was negative at the end of Stage 3. Expression of SOX2 protein was weak and where present was detected solely within the cytoplasm of the cells which provided further evidence of the loss of pluripotency. The presence of other developmentally immature cells was assessed by immunostaining for MIXL1, BRACHYURY and SOX17 (FIG. 12). Protein expression of all three markers was faint at the end of stage 3 and restricted to the cytoplasm, suggesting that most cells had differentiated beyond these intermediary stages. This contrasts with spontaneous embryoid body-derived differentiating cells which yielded a much more heterogeneous cell population (FIG. 13). Similarly, immunostaining for GATA4 and SOX7, characteristic of definitive and visceral endoderm respectively, was markedly reduced by the end of Stage 3 (FIG. 12). Despite low expression of SOX7 transcript (FIG. 3o), there was no nuclear localization of the protein. The mesodermal marker, PDGFRI3, was absent at the end of Stage 3 and FLK1 was considerably reduced (FIG. 12). PDGFR3 (FIG. 12) was expressed cells at the end of Stage 3. Immunostaining for SOX9, CD44, and Collagen II associated with chondrocytes (FIG. 5 and FIG. 12), showed that cells at the end of Stage 3 had a chondrocyte-like phenotype. Although basal SOX9 gene expression was detected in pluripotent hES cells and at the end of Stage 1 (FIG. 3s), no SOX9 protein was detected within pluripotent hES cell cultures (FIGS. 5a-c). In contrast at the end of Stage 3 SOX9 was highly expressed and the majority of positive cells showed nuclear localization (FIGS. 5d-i). CD44 was detected on Stage 3 cells as punctate immunostaining at the cell surface (FIG. 12). Collagen type II was also abundant, providing a clear indication that the cells had developed a chondrocyte-like phenotype (FIGS. 5j-p).

### Flow Cytometry for SOX9, CD44 and CD105 in Cells at the End of the Protocol.

Flow cytometry was used to quantify the proportion of immuno-positive cells within the final population SOX9 protein was expressed by 74.8%±3.1 (N=4) of the culture at the end of Stage 3 (FIGS. 6a, b) though the proportion of cells expressing the hyaluronan receptor CD44 (34.9%±2.1 (N=4) was much lower than the proportion expressing SOX9 (FIGS. 6c, d). We also analyzed expression of CD105 (characteristic of undifferentiated mesenchymal stem cells (MSC) as shown in FIG. 10) (Pittenger, M. F. et al. Science 284, 143-147 (1999)). CD105 was expressed on only 12.7%±2.5 (N=4) of the cells at the end of the protocol (FIGS. 6e, f). The rounded morphology, expression of SOX9, CD44 and collagen type II together with the lack of CD105 indicates that the protocol produces cells more developmentally advanced towards chondrocytes than MSCs.

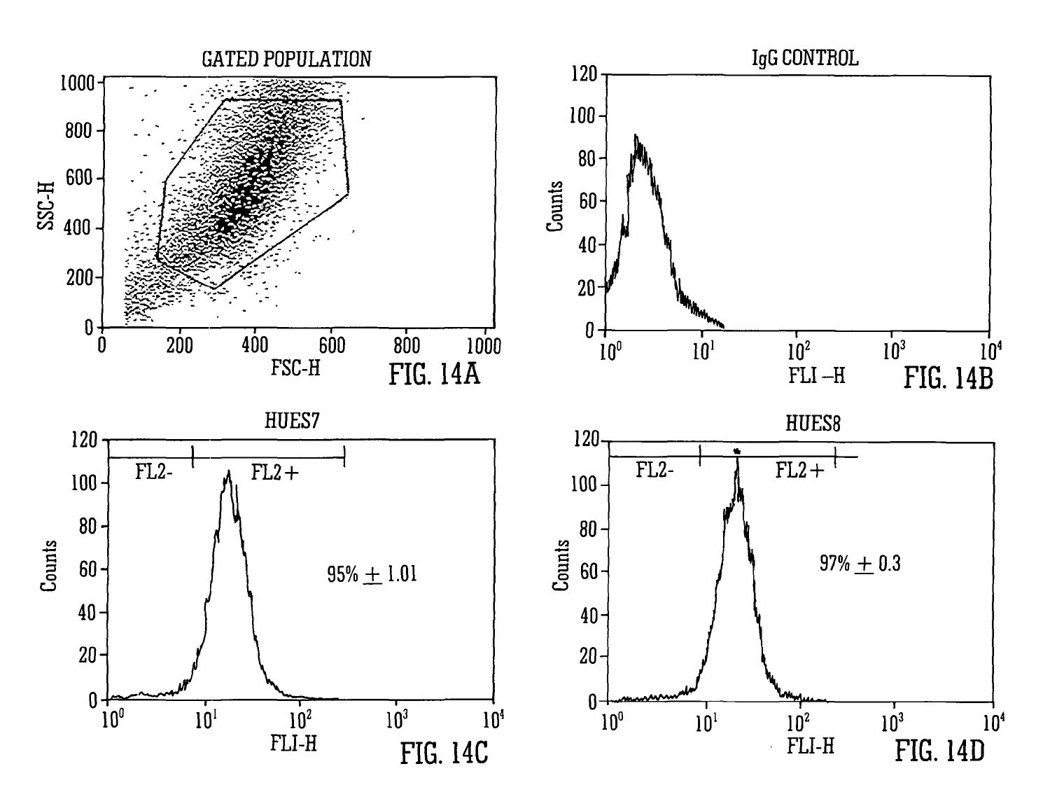

The protocol was developed throughout with HUES1 cells, but has now been tested with HUES7 and HUES8 cells. These both showed very similar cell morphology and characteristics to HUES1 when cultured through the protocol and interestingly they both showed a higher proportion of SOX9 positive cell progeny at the end of the protocol (Stage 3), HUES7 (95%±1.01) (N=4) and HUES8 (97%±0.3) (N=4) (FIG. 14).

### Discussion

A chemically-defined protocol for the directed differentiation of hES cells towards chondrocytes has been developed. The differentiation protocol was divided into stages to incorporate the transient enrichment of mesodermal intermediate cell populations that are known to precede chondrogenesis during development. This 3-stage protocol is highly efficient, resulting in a chondrogenic population of which, in 3 different hES lines, 74%-97% of cells express the chondrocyte associated transcription factor, SOX9. During the differentiation process the cells acquired a rounded chondrocyte-like morphology, expressed the hyaluronan cell surface receptor CD44 and formed 3D aggregates in which collagen type II and a sGAG-rich matrix were deposited. The development of cell-based therapies for the treatment of articular cartilage defects has extensively focused on the application of adult stem cells derived primarily from mesenchymal tissues. This has suggested that chondrogenic differentiation of adult stem cells results in a hypertrophic phenotype, indicative of differentiation along an endochondral ossification route (Barry, F., Exp Cell Res 268, 189-200 (2001), Pelttari, K. Injury 39 Suppl 1, S58-65 (2008)). In contrast to hMSC chondrogenic differentiation there was no expression of COL10A1 (associated with hypertrophic) detected at any stage during the protocol. Further to this the proliferative and chondrogenic potential of hMSC has been shown to be compromised by the age of the donor and the incidence of musculoskeletal diseases such as osteoarthritis (Coipeau, P. et al. Cytotherapy 11, 584-594 (2009) Murphy, J. M. et al. Arthr Rheum 46, 704-713 (2002)). Hence it is believed that embryonic stem cells are a more developmentally appropriate source for the generation of clinically relevant chondrogenic cells.

The protocol started with a feeder-free culture system which not only eliminated undefined bio-active molecules secreted by feeder cells, but also created a more homogeneous culture from which differentiation proceeded. It is postulated that this allowed for more uniform exposure to growth factors, nutrients and oxygen tension, all of which can influence hES cell fate decisions (Sachlos, E. & Auguste, D. T. Biomaterials 29, 4471-4480 (2008)) and hence efficient differentiation towards chondrocytes. A 2D culture format was adopted which has more potential than 3D embryoid bodies for producing a high-yield, scalable protocol. Culture in 2D also facilitated cell proliferation (aided by the pleiotrophic growth factor, FGF2) (Baxter, M. A. et al. Stem Cell Res 3, 28- 38 (2009)). The development of hES derived cells for clinical therapies requires cell expansion for transplantation and appropriately the protocol achieved an 8.5-fold increase in cell number between days 0 and 12.

We found that a proprietary anti-oxidant supplement (B27) and a mix of insulin, transferrin and selenium (ITS) supported cell metabolism and enhanced cell survival. However, it is of interest that, whilst this base medium conferred some advantages, it was unable to support prolonged culture on its own. From day 4 of the protocol we added a low concentration of NT4, to maintain hES cell survival (Pyle, Nature Biotechnol 24, 344-350 (2006)).

Furthermore, it is likely that FGF2 added from day 2 also contributed to cell survival. In the initial step, cells were cultured on fibronectin as for hES cell maintenance. As culture proceeded fibronectin became dispensable and cells could be progressively passaged onto a gelatin substrate. We postulate that as differentiation proceeded, the cells deposited and assembled a more complex extracellular matrix, which may be important in maintaining cell survival.

In the first stage of differentiation pluripotent hES cells were directed to a bi-potent mesendoderm intermediate population, characterized by upregulation of E-cadherin, goosecoid and brachyury. In assessing gene expression it must be born in mind that we artificially designate the end of each stage as a single time point in a continuous differentiation programme. However, the peaks in transient expression will differ between genes and may occur on either side of times of analysis. Interestingly, the transient upregulation of the mesendoderm marker MIXL1 was highest at the end of the stage 2 when GSC, ECAD and FOXA2 had all been downregulated. It is likely that MIXL1 transcription was suppressed in the presence of goosecoid and so increased only when after the latter had decreased, reflecting the expression observed during in vivo development.

It is considered that optimization of Stage 1, priming the cells for later signaling cues, was the most crucial step in developing an efficient differentiation protocol, since in preliminary experiments it had the most influence over the final cellular phenotype. Particularly significant was the concentration of activin. When high concentrations of activin were used to drive differentiation to mesendoderm significant levels of endoderm associated genes, specifically, SOX17, FOXA2 and GATA4, remained at the end of the protocol. This was despite including insulin, an inhibitor of definitive endoderm differentiation in the medium McLean, A. B. et al. Stem Cells 25, 29-38 (2007)), and two subsequent stages of differentiation with the pro-mesoderm/pro-chondrogenic cytokines, BMP4 and GDF5. This potent effect of activin concentration on cell lineage specification in vitro has been described previously and may reflect the morphogenic gradients along the anterior-posterior axis of the primitive streak: activin concentration highest anteriorly where cells become specified to endoderm cell lineages (Sumi, T., Development 135, 2969-2979 (2008)). Cells from the posterior region of the primitive streak which differentiate into mesoderm cells are exposed to lower concentrations of activin. Conversely, D'Amour et al (2006) demonstrated that low concentrations of activin within Stage 1 of their differentiation protocol, reduced the proportion of insulin-positive cells. Following this developmental paradigm we applied wnt3a for 3 days and added BMP4 on day 3 of Stage 1. Both of these cytokines are prominent within more posterior regions of the primitive streak and direct cells towards mesodermal cell lineages. (Ng, E. S. et al. The primitive streak gene MixI1 is required for efficient haematopoiesis and BMP4-induced ventral mesoderm patterning in differentiating ES cells. Development 132, 873-884 (2005). Park, C. et al. A hierarchical order of factors in the generation of FLK1- and SCL-expressing hematopoietic and endothelial progenitors from embryonic stem cells.Development 131, 2749-2762 (2004). Zhang, P. et al. Short-term BMP-4 treatment initiates mesoderm induction in human embryonic stem cells. Blood 111, 1933-1941 (2008)).

In Stage 2 of the protocol the mesendoderm-like population was directed towards a mesodermal phenotype by continued treatment with BMP4, involved in particularly posterior mesoderm patterning. (Ng, E .S. et al. The primitive streak gene MixI1 is required for efficient haematopoiesis and BMP4-induced ventral mesoderm patterning in differentiating ES cells. Development 132, 873-884 (2005). Park, C. et al. A hierarchical order of factors in the generation of FLK1- and SCL-expressing hematopoietic and endothelial progenitors from embryonic stem cells. Development 131, 2749-2762 (2004). Zhang, P. et al. Short-term BMP-4 treatment initiates mesoderm induction in human embryonic stem cells. Blood 111, 1933-1941 (2008)). Inhibition of FGF-signaling has been shown to attenuate BMP4-mediated mesoderm specification and anterior-posterior mesoderm patterning (Faloon, P. et al. Basic fibroblast growth factor positively regulates hematopoietic development. Development 127, 1931-1941 (2000)) so we continued supplementation with FGF2. However we removed activin from the culture to increase efficiency of differentiation to mesoderm as confirmed by the up-regulation of PDGFR□, FLK1 and CXCR4 (Ema, M., Takahashi, S. & Rossant, J. Deletion of the selection cassette, but not cis-acting elements, in targeted Flk1-lacZ allele reveals Flk1 expression in multipotent mesodermal progenitors. Blood 107, 111-117 (2006) (Era, T. et al. Multiple mesoderm subsets give rise to endothelial cells, whereas hematopoietic cells are differentiated only from a restricted subset in embryonic stem cell differentiation culture. Stem Cells 26, 401-411 (2008) (Zhang, P. et al. Short-term BMP-4 treatment initiates mesoderm induction in human embryonic stem cells. Blood 111, 1933-1941 (2008) (Kataoka, H. et al. Expressions of PDGF receptor alpha, c-Kit and Flk1 genes clustering in mouse chromosome 5 define distinct subsets of nascent mesodermal cells. Dev Growth Diff 39, 729-740 (1997)) in Stage 2. FOXA2, SOX17 and GATA4 associated with definitive endoderm were all significantly down-regulated at the end of Stage 2 suggesting that the majority of cells had differentiated towards mesoderm, while the expansion of endoderm-lineage cells within the Stage 1 population had been prevented. Whether the latter cells die, or are redirected, cannot be ascertained from this study.

As well as mediating mesoderm germ layer specification, BMPs are prominent regulators of chondrogenesis; particularly, BMP4 is involved in instructing uncommitted mesenchymal cells to become chondroprogenitors Hatakeyama, Y., Nguyen, J., Wang, X., Nuckolls, G. H. & Shum, L. Smad signaling in mesenchymal and chondroprogenitor cells. J bone Joint Surg Am 85-A Suppl 3, 13-18 (2003)). During Stage 2 we observed the self-aggregation of cells into 3D clusters reminiscent of the mesenchymal condensations that form at the initiation of chondrogenesis. In keeping with this, genes associated with chondrogenic differentiation, particularly, SOX9 and its downstream target collagen II, increased at the end of Stage 2.

In Stage 3 we aimed to enrich for a chondrocyte-like population. Whilst treatment with BMP4 is regarded as chondrogenic and has been shown previously to initiate the formation of mesenchymal condensations (Hatakeyama, Y., Nguyen, J., Wang, X., Nuckolls, G. H. & Shum, L. Smad signaling in mesenchymal and chondroprogenitor cells. J bone Joint Surg Am 85-A Suppl 3, 13-18 (2003), Hatakeyama, Y., Tuan, R.S. & Shum, L. Distinct functions of BMP4 and GDF5 in the regulation of chondrogenesis. J Cell Biochem 91, 1204-1217 (2004)), the effect of BMP4 alone also resulted, here and elsewhere, in the expression of the hemangioblast markers FLK1 and CXCR4 (Era, T. et al. Multiple mesoderm subsets give rise to endothelial cells, whereas hematopoietic cells are differentiated only from a restricted subset in embryonic stem cell differentiation culture. Stem Cells 26, 401-411 (2008) Kataoka, H. et al. Expressions of PDGF receptor alpha, c-Kit and Flk1 genes clustering in mouse chromosome 5 define distinct subsets of nascent mesodermal cells. Dev Growth Diff 39, 729-740 (1997)). Because of this, we added a related member of the TGF superfamily, GDF5 (Hatakeyama, Y., Nguyen, J., Wang, X., Nuckolls, G. H. & Shum, L. Smad signaling in mesenchymal and chondroprogenitor cells. J bone Joint Surg Am 85-A Suppl 3, 13-18 (2003), Hatakeyama, Y., Tuan, R. S. & Shum, L. Distinct functions of BMP4 and GDF5 in the regulation of chondrogenesis. J Cell Biochem 91, 1204-1217 (2004), which has a prominent role during chondrogenesis, and in maintenance of the chondrogenic phenotype in mature articular cartilage (Pacifici, M., Koyama, E. & Iwamoto, M. Mechanisms of synovial joint and articular cartilage formation: recent advances, but many lingering mysteries. Birth Defects Res C Embryo Today 75, 237-248 (2005)) and has been shown to be more potent than BMP4 at initiating mesenchymal condensations and subsequent cartilage nodule formation (Hatakeyama, Y., Tuan, R. S. & Shum, L. Distinct functions of BMP4 and GDF5 in the regulation of chondrogenesis. J Cell Biochem 91, 1204-1217 (2004)).

Analysis of the 3D aggregates formed at the end of Stage 3 showed that cells had assembled a cartilage-specific collagen type II and sGAG-rich matrix, characteristic of a cartilagenous tissue (Hardingham, T. E. Articular Cartilage in Oxford Textbook of Rheumatology. (ed. M. P. Isenberg, P. J. Maddison, P. Woo, D. Glass, F. C. Breedveld) 325-334 (Oxford University Press, Oxford; 2004)). Cells within aggregates expressed SOX9, which was prominently localized to the cell nucleus, an indication of functionally activity. Flow cytometry showed that 74% (and 95-97% of HUES 7 and 8) of the final population was positive for SOX9 and this may reflect inherent differences between genetically distinct cell lines in their tendency to form chondrogenic cells, as previously reported for hES cells (Osafune, K. et al. Marked differences in differentiation propensity among human embryonic stem cell lines. Nature Biotechnol 26, 313-315 (2008)) or to respond to the in vitro conditions. It is possible that over 95% of all three HUES lines would become SOX9-positive given sufficient time in culture). It is possible that some of the unlabeled cells of the final population represented progenitors of other mesenchymal-lineages and this was clearly much less in HUES7 and HUES8 cells. Whilst cells within the final Stage 3 culture, aggregate and acquire a rounded morphology, a proportion of the cells remain adherent to the plastic and the effect of 2D substrate adhesion upon the chondrocyte phenotype has previously been demonstrated to induce actin stress fiber formation and a loss of SOX9 expression (Tew, S. R. & Hardingham, T. E. Regulation of SOX9 mRNA in human articular chondrocytes involving p38 MAPK activation and mRNA stabilization. J Biol Chem 281, 39471-39479 (2006)). This effect may variably influence the number of SOX9 positive cells. The proportion of cells in the final population HUES1 cells expressing the hyaluronan cell surface receptor CD44 (34%) was less than SOX9. In mature cartilage CD44 provides a link between the chondrocyte and the pericellular matrix through its interaction with hyaluronan. CD44 expression has been shown to be responsive to the amount of matrix surrounding the cell (Grover, J. & Roughley, P. J. Expression of cell-surface proteoglycan mRNA by human articular chondrocytes. Biochem J 309 (Pt 3), 963-968 (1995)) and hence the lower proportion of cells expressing CD44 might reflect the immaturity of the ECM formed within the aggregates. We therefore suggest that these cells are immature chondrocytes.

At the end of Stage 3 the cells did not express the pluripotency markers OCT4 and NANOG, which is an important safety consideration for translation of the protocol towards a clinical therapy, since un-differentiated hES cells might give rise to teratomas in vivo (Liew, C. G. et al. Human embryonic stem cells: possibilities for human cell transplantation. Ann Med 37, 521-532 (2005). In contrast to undirected spontaneous differentiation, the cells produced in this protocol had minimal expression of proteins associated with developmental intermediate cell populations, or non-target cell types. This suggests that the protocol efficiently directs hES cell differentiation towards chondrocytes. The success of the protocol is likely to occur by a combination of directing hES cell differentiation through each developmental stage, as well as providing a culture environment in which target cells thrive and non-target cell types are lost. The prime advantage of the protocol is its efficiency in deriving chondrocytic cells from hES cells which was achieved with 3 genetically different hES cell lines. Most importantly the protocol is carried out in a fully chemically defined and scalable system, making it an appropriate methodology for the more detailed analysis of chondrogenesis during mammalian development and a basis for the production of chondrocytes suitable for translation to future clinical therapies.

### Methods

### Cell Culture

2D monolayer culture of hES Cells All cell culture media and supplements were purchased from Gibco®, Invitrogen, Paisley, UK unless otherwise stated. Human embryonic stem (hES) cell lines HUES1, HUES7 and HUES8 (passage 24-42) Cowan, C. A. et al. New Engl J Med 350, 1353-1356 (2004) http://www.mcb.harvard.edu/melton/hues/) were cultured for bulk up on mitomyci n-C-inactivated MF1×CD1 mouse embryonic fibroblasts (MEFs) in hES cell medium (Knockout™ DMEM, 20% (vol/vol) Knockout™ Serum Replacement, 1% (vol/vol) ITS, 1% (vol/vol), non-essential acids, 2 mM L-glutamine, 50 U/ml penicillin 50 μg/ml streptomycin, 90 μM β-mercaptoethanol supplemented with 10 ng/ml FGF2 (Biosource™, Invitrogen). Feeder-free hES cell culture was carried out as described previously (Baxter, M. A. et al. Analysis of the distinct functions of growth factors and tissue culture substrates necessary for the long-term self-renewal of human embryonic stem cell lines. Stem Cell Res 3, 28-38 (2009)). Briefly, hES cells were lifted from MEF feeders with trypsin (PAA Laboratories Ltd., Yeovil, UK), centrifuged at 720×g for 3 minutes before being, resuspended in feeder-free culture medium (DMEM:F12, 0.1% (wt/vol) BSA (Sigma, Poole, UK), 2 mM L-glutamine, 1% (vol/vol) non-essential acids, 2% (vol/vol) B27, 1% (v/v) N2 liquid supplement, 90 μM 3-mercaptoethanol, 10 ng/ml activin-A (Peprotech, London, UK), 40 ng/ml FGF2, 2 ng/ml neurotrophin-4 (Peprotech). hES cells were replated onto fibronectin-coated (50 μg/ml) (Millipore, Watford, UK) tissue culture plastic. Initially feeder-free cultures were established by sub-culturing confluent hES cell cultures at a ratio of 1:1 to achieve approximately 90% plating efficiency. Once established, confluent feeder-free cultures were sub-cultured by trypsin passage and re-plated onto fresh fibronectin substrate at a cell density of 5×10⁴ cells/cm², a ratio of approximately 1:4. Feeder-free hES cells were cultured over at least 3 passages to ensure the removal of all MEF cells. For directed differentiation studies, hES cells were cultured on tissue culture plastic, coated with appropriate matrix substrates in a basal medium (DMEM:F12, 2 mM L-glutamine, 1% (vol/vol) ITS, 1% (vol/vol) non-essential acids, 2% (vol/vol) B27, 90 μM β-mercaptoethanol) supplemented with appropriate growth factors as described in the results section and in table 1 as follows: Wnt3a (R&D Systems, Abingdon, UK), human follistatin 300 (Sigma), BMP4, GDF5 (Peprotech, London, UK). Cell counts were performed at each passaging event in order to quantify the expansion of the cell culture during directed differentiation. For immunostaining at the end of stage 3, cells were sub-cultured on day 12 onto glass coverslips coated with gelatin. Embryoid body (EB) formation and culture Feeder-free HUES1 cells were detached from tissue culture plastic by incubation with collagenase IV for 10 minutes at 37° C., 5% CO₂. Small clumps of cells were resuspended in DMEM (DMEM, 10% (v/v) FBS, 110 μg/ml sodium pyruvate, 100 U/ml penicillin, 100 μg/ml streptomycin) and plated as a suspension culture in bacteriological grade petri dishes. After 7 days, EBs were dissociated by trypsin digestion and plated on either FBS-coated tissue culture plastic or glass coverslips (for immunostaining) for the required number of days.

### Gene Expression Analysis

RNA purification Total RNA was extracted from monolayer cell cultures using Qiashredder™ and RNeasy™ mini kits (Qiagen, Crawley, UK) according to the manufacturer's instructions and treated with 10 U/μl RNA sample DNasel (Invitrogen). Total RNA (1 μg per 25 μl reaction) was reverse transcribed using 200 U M-MLV primed with 0.5 μg random hexamer oligonucleotides (Promega, Southampton, UK). Quantitative PCR analysis PCRs were carried out on a MJ Research Opticon 2 using Sybr Green I detection (Eurogentec, Seraing, Belgium) according to the manufacturer's instructions. Gene specific primers were designed using Applied Biosystems Primer Express software and used at a final concentration of 300 nM. Relative expression levels were normalized to GAPDH and calculated using the 2^{-ΔCt} method (Livak, K. J. & Schmittgen, Method. Methods 25, 402-408 (2001)). All primers were purchased from Invitrogen and are described in Table 3.

### Immunofluorescence/Immunohistochemistry and Histological Analysis

For sGAG detection, fixed cells were stained in 0.1% (wt/vol) safranin O (in 0.1% (vol/vol) in acetic acid) for 5 minutes at room temperature. For determining the specificity of safranin O for sGAG, some cultures were incubated with 1 unit/ml chondroitinase ABC (Sigma) in chondroitinase buffer (50 mM Tris (pH 8.0), 60 mM sodium acetate, 0.02% (wt/vol) BSA) for 30 minutes at 37° C. prior to safranin O staining. For determining the frequency of safranin O positive cell clusters at Stage 2 and cell aggregates at Stage 3, cultures were imaged on an Olympus CKX41 microscope with Olympus U-CMAD3 camera. Cell clusters/aggregates which contained more than 12 cells were counted over predetermined independent fields of view (0.35 mm² (700 μm×500 μm)) chosen at random. The average of ten fields was taken for each of four replicates. Immunofluorescence For immunolocalization, samples were blocked (0.1% (wt/vol) BSA, 10%(vol/vol) serum of animal in which secondary was raised, in PBS) for 1 hour, incubated at 4° C. for 16 hr with primary antibody (Table 2) or irrelevant primary and for 1 hour in appropriate secondary antibody (donkey anti-mouse IgG Alexa 488 or donkey anti-goat IgG Alexa 488) both diluted (1:250) in blocking buffer. Cells were counterstained with 300 nM 4′-6-diamidino-2-phenylindole (DAPI) for 5 minutes before mounting with Vectorshield (Vector Laboratories, Inc., Peterborough, UK). For immunofluorescence of COLLAGEN II protein, cell cultures were treated with chondroitinase ABC prior to blocking. Appropriate immunological controls were carried out in parallel. Antibodies were purchased as described in Table 2.

### Biochemical Analysis

Preparation of samples Cell cultures were digested in 10 units/ml papain in papain buffer (0.1M sodium acetate, 2.4 mM ethylenediaminetetraacetic acid, 5 mM L-cysteine, pH 5.8) (all from Sigma) in 50 μl/cm² of culture area for 2 hours at 60° C. Quantification of DNA The amount of DNA in papain-digested samples was determined using a Quant-iT™ PicoGreen® ds DNA kit (Invitrogen) according to the manufacturer's instructions. 1,9-dimethylmethylene blue (DMMB) assay for sGAG One hundred μl MMB solution (16 mg/L 1,9-dimethylmethylene blue, 40 mM glycine, 40 mM NaCl, 9.5 mM HCl, (pH3.0)) was added to papain-digested samples (40 μl) and the absorbance (A₅₄₀) of the resultant solution read immediately. The amount of sGAG in each sample was calculated against a known standard of shark chondrondroitin (Sigma).

### Flow Cytometry

Cells were detached and digested to a single cell suspension using trypsin. For labeling of intracellular antigens, cells were fixed in ice-cold methanol for 10 minutes at -20° C. and further permeabilized by incubation with 1% (wt/vol) BSA, 0.5% (vol/vol) triton-x-100 in PBS for 15 minutes. Cells were incubated with primary antibody (goat anti-human SOX-9, (8 μg/ml) diluted in ice-cold buffer (10% (vol/vol) FBS, 1% (wt/vol) sodium azide in PBS) overnight at 4° C. For labeling of cell surface antigens, cells were incubated with primary antibody (mouse anti-human CD44 (50 μg/ml), goat anti-human Endoglin/CD105 (5 μg/ml) in ice-cold buffer (2% (vol/vol) FBS, 0.1% (wt/vol) sodium azide in PBS) for 2 hours. Cells were incubated with appropriate secondary antibodies (donkey anti-mouse IgG Alexa 488 or donkey anti-goat IgG Alexa 488) diluted to 1:250. Cell labeling was analyzed using a Cell Lab Quanta™ SC MPL flow cytometer with software version 1.0 (Beckman Coulter, High Wycombe, UK).

### Statistical Analysis

Data sets were analyzed for normal distribution using the one-sample Kolmogorov-Smirnov test. Normally distributed data was analyzed by parametric two-sample t-test. Data which was not normally distributed was analyzed by an equivalent non-parametric test.

### Supplementary Methods

### Optimization of the Directed Differentiation Protocol

The differentiation protocol described and characterized in the main article was developed through empirical and systematic optimization of each stage of differentiation. Preliminary protocols were carried out on the embryonic stem cell line HUES7 with directed differentiation driven by culturing the cells in base medium (described in the main methods section) supplemented with combinations of exogenous growth factors. Differentiation to our target chondrogenic cells was assessed by semi-quantitative PCR (sqPCR) for expression of target and non-target genes associated with mesendodermal (MIXL1), mesodermal (brachyury, PDGFRβ, FLK1), endodermal (SOX17, FOXA2, GATA4, AFP), neurectodermal (SOX1, PAX6, FGF5) and chondrogenic (SOX9, CD44, Collagen II). To assess the progression and efficiency of our directed differentiation over random spontaneous differentiation, sqPCR was carried out on cDNA generated from outgrowths of embryoid bodies which had been cultured over equivalent lengths of time. Progressive refinement of our pilot protocols was carried out by modifying the growth factors applied, the concentrations at which they were added, and the kinetics of application. Criteria for successful refinement of our protocol was the elimination of expression of genes associated with non-target cell lineages whilst establishing and maintaining maximal expression of genes associated with chondrogenic differentiation. The ranges of each growth factor concentration and days of application which were assessed are listed below:

Wnt3a: 25 ng/ml; day 1 alone, days 1-2 inclusive, days 1-3 inclusive

Activin-A: Assayed at concentrations of 100 ng/ml-10 ng/ml over days 1-8

BMP4: Assayed at concentrations of 10 ng/ml-40 ng/ml over days 3-10

NT4: Assayed at concentrations of 0-2 ng/ml added from day 4-14

Follistatin: Assayed at concentrations of 50 ng/ml-100 ng/ml over days 4-7

FGF2: Assayed at concentrations of 10 ng/ml-20 ng/ml over days 2-14

GDF5: Assayed at concentrations of 10 ng/ml-40 ng/ml over days 9-14

hMSC Culture and Characterization of Chondrogenic Differentiation

### Cell Culture

Culture and chondrogenic differentiation of hMSC hMSC were derived from human bone marrow mononuclear cell preparations (Lonza, Wokingham, UK) and cultured as previously described⁸¹. At Passage 3 hMSC were formed into cell aggregates (500,000 cells per aggregate) and cultured for up to 14 days in chondrogenic medium (high-glucose Dulbecco's modified Eagle's medium (DMEM), 2 mM I-glutamine, 110 μg/ml sodium pyruvate, 100 U/ml penicillin, 100 μg/ml streptomycin, 50 μg/ml ascorbic acid 2-phosphate, μg/l I-proline, 100 nM dexamethasone, 100 ng/ml TGF-3, 1% (vol/vol) ITS+1 supplement. Cells were centrifuged into aggregate cultures at day 0.

### Gene Expression Analysis

Total RNA was extracted using Tri-reagent (Sigma-Aldrich) from cell aggregates ground using Molecular Grinding Resin (Geno Technology Inc., St. Louis, http://www.gbiosciences.com). DNase-treatment, reverse-transcription and gene expression analysis was as described in the methods section of the main article.

TABLE 1
Protocol for stages 1-3 of the differentiation regime for hES cells. hES cells were fed daily in base
medium (materials and methods) supplemented with growth factors at the stated concentrations.
Differentiating cultures were expanded by passage with trypsin.

| | | CULTURE AREA | MATRIX | WNT3A | ACTIVIN-A | FGF2 | BMP4 | FOLLISTATIN | GDF5 | NT4 |
|---|---|---|---|---|---|---|---|---|---|---|
| STAGE | DAY | (CM²) | SUBSTRATE | (ng/ml) | | | | | | |
| 1 | 1 | 9.62* | FN | 25 | 50 | | | | | |
| 1 | 2 | 9.62 | FN | 25 | 25 | 20 | | | | |
| 1 | 3 | 9.62 | FN | 25 | 10 | 20 | 40 | | | |
| 2 | 4 | 9.62 | FN | | | 20 | 40 | 100 | | 2 |
| 2 | 5 | 48.11 | FN | | | 20 | 40 | 100 | | 2 |
| 2 | 6 | 48.11 | FN | | | 20 | 40 | 100 | | 2 |
| 2 | 7 | 48.11 | FN | | | 20 | 40 | 100 | | 2 |
| 2 | 8 | 192.44 | FN: GEL | | | 20 | 40 | | | 2 |
| 3 | 9 | 192.44 | FN: GEL | | | 20 | 20 | | 20 | 2 |
| 3 | 10 | 192.44 | FN: GEL | | | 20 | 20 | | 20 | 2 |
| 3 | 11 | 192.44 | FN: GEL | | | 20 | | | 40 | 2 |
| 3 | 12 | 288.66 | GEL | | | 20 | | | 40 | 2 |
| 3 | 13 | 288.66 | GEL | | | 20 | | | 40 | 2 |

The ratio of expansion for each passage described as an increase in culture area (cm²),
*= the culture area of a 35 mm tissue culture dish. Tissue culture dishes were coated with fibronectin, fibronectin/gelatin mixed at a ratio of 50:50 or gelatin.
FN = fibronectin,
GEL = gelatin,
NT4 = neurotrophin-4.

TABLE 2
Antibodies used for immunofluorescence and flow cytometry. The following
table presents information on the antibodies used in the characterisation of protein
expression. The supplier and catalogue number is provided as well as the final
concentration of antibody used for each application.

| | | Final concentration | |
|---|---|---|---|
| Protein | Antibody | (μg/ml) | Supplier |
| Brachyury | Goat IgG anti-human brachyury (AF2085) | 4 μg/ml | R&D Systems |
| Collagen II | Goat IgG anti-human collagen type II (clone | 4 μg/ml | Insight |
| | N-19) (sc-7764) | | Biotechnology |
| CD44 | Monoclonl IgG2ₐ anti-human CD44H | 10 μg/ml | R&D Systems |
| | (BAA10) | (immunofluorescence) | |
| | | 50 μg/ml (flow cytometry) | |
| CD105 | Goat IgG anti-human Endoglin/CD105 | 5 μg/ml | R&D Systems |
| | (AF1097) | | |
| CXCR4 | Monoclonal Rat IgG2ₐ anti-mouse CXCR4 | 5 μg/ml | R&D Systems |
| | (MAB21651) | | |
| E-CADHERIN | Mouse IgG2a anti-human E-Cadherin (13- | 20 μg/ml | Invitrogen |
| | 5700) | | |
| FOXA2 | Goat IgG anti-human HNF3β/FOXA2 | 5 μg/ml | R&D Systems |
| | (AF2400) | | |
| GATA4 | Goat IgG anti-human GATA-4 (AF2606) | 4 μg/ml | R&D Systems |
| MIXL1 | Goat IgG anti-MIXL (ab57854) | 5 μg/ml | Abcam |
| GOOSECOID | Goat IgG anti-human goosecoid (AF4086) | 10 μg/ml | R&D Systems |
| NANOG | Goat IgG anti-human Nanog (AF1997) | 2 μg/ml | R&D Systems |
| OCT4 | Monoclonal IgG1 anti-human Oct-3/4 | 2.5 μg/ml | BD Biosciences |
| | (611202) | | Pharmingen ™ |
| PDGFRα | Goat IgG anti-human PDGFRα (AF-307-NA) | 1 μg/ml | R&D Systems |
| PDGFRβ | Monoclonal IgG₁ anti-human PDGFRβ | 5 μg/ml | R&D Systems |
| | (MAB385) | | |
| SOX1 | Goat IgG anti-human SOX1 (AF3369) | 2 μg/ml | R&D Systems |
| SOX2 | Monoclonal IgG₂ₐ anti-human/mouse | 5 μg/ml | R&D Systems |
| | (MAB2018) | | |
| SOX7 | Monoclonal IgG_{2b} anti-human SOX7 | 5 μg/ml | R&D Systems |
| | (MAB2766) | | |
| SOX9 | Goat IgG anti-human SOX9 (AF3075) | 4 μg/ml | R&D Systems |
| | | (immunofluorescence) | |
| | | 8 μg/ml (flow cytometry) | |
| SOX17 | Goat IgG anti-human SOX17 (AF1924) | 5 μg/ml | R&D Systems |
| FLK1 | Monoclonal IgG₁ anti-human VEGF R2 | 2.5 μg/ml | R&D Systems |
| | (KDR) (MAB3571) | | |
| Secondary antibody | Alexa Fluor ® 488 donkey anti-goat IgG | 1:250 dilution from | Invitrogen |
| | (H + L) (A-11056) | purchased stock | (molecular |
| | | | probes) |
| Secondary antibody | Alexa Fluor ® 488 donkey anti-mouse IgG | 1:250 dilution from | Invitrogen |
| | (H + L) (A-21202) | purchased stock | (molecular |
| | | | probes) |
| Immunological | Normal goat IgG (sc-2028) | As for appropriate primary | Insight |
| control antibody | | antibody | Biotechnology |
| Immunological | Normal mouse IgG (sc-2025) | As for appropriate primary | Insight |
| control antibody | | antibody | Biotechnology |

TABLE 3
Gene-specific oligonucleotide primers used in quantitative PCR.

| | Primer sequences | GenBank accession number (and base |
|---|---|---|
| Gene | (forward and reverse, 5′-3′) | pairs covered by primers) or reference |
| αFP | CCAACAGGAGGCCATGCT | NM_001134 |
| | GAGAATGCAGGAGGGACATATGT | (1564-1627) |
| Aggrecan | TCGAGGACAGCGAGGCC | 78 |
| | TCGAGGGTGTAGCGTGTAGAGA | |
| Albumin | GACTTGCCAAGACATATGAAACCA | NM_000477 |
| | TTGGCATAGCATTCATGAGGAT | (1188-1260) |
| Brachyury | GGGTCCACAGCGCATGAT | NM_003181 |
| | TGATAAGCAGTCACCGCTATGAA | (1021-1089) |
| CBFA1 | GCCTTCAAGGTGGTAGCCC | NM_004348.3 |
| | CGTTACCCGCCATGACAGTA | (773-839) |
| CD44 | ACGTGGAGAAAAATGGTCGCT | NM_000610 |
| | TTGAAAGCCTTGCAGAGGTCA | (538-604) |
| CXCR4 | CGCCTGTTGGCTGCCTTA | NM_003467 |
| | ACCCTTGCTTGATGATTTCCA | (842-915) |
| Collagen II | GGCAATAGCAGGTTCACGTACA | 78 |
| | CGATAACAGTCTTGCCCCACTT | |
| Collagen X | CAAGGCACCATCTCCAGGAA | 78 |
| | AAAGGGTATTTGTGGCAGCATATT | |
| E-Cadherin | GCATTGCCACATACACTCTCTTCT | NM_004360 |
| | AATCTCCATTGGATCCTCAACTG | (795-868) |
| FGF5 | GCGATGTCAAAAAAAGGAAAACTC | NM_004464 |
| | TCTTGAAAACGCTCCCTGAAC | (664-740) |
| Flk1 | TGATGCCAGCAAATGGGAAT | NM_002253 |
| | CCACGCGCCAAGAGGCTTA | (2769-2831) |
| FOXA2 | TTCAGGCCCGGCTAACTCT | NM_021784 |
| | AGTCTCGACCCCCACTTGCT | (1590-1656) |
| GAPDH | ATGGGGAAGGTGAAGGTCG | 78 |
| | TAAAAGCAGCCCTGGTGACC | |
| GATA4 | CCTCCTCTGCCTGGTAATGACT | NM_002052 |
| | CGCTTCCCCTAACCAGATTG | (1933-209) |
| Goosecoid | GATGCTGCCCTACATGAACGT | NM_173849 |
| | GACAGTGCAGCTGGTTGAGAAG | 549-619 |
| HNF1α | TCCCATCCCCAGCGATT | NM_000545 |
| | CTTGGGAACAAATACAGGAAAGCT | (2578-2643) |
| HNF4α | AGATGAGCCGGGTGTCCAT | NM_000457 |
| | CGATCTGCAGCTCCTGGAA | (868-933) |
| L-SOX5 | ATCCCAACTACCATGGCAGCT | 79 |
| | TGCAGTTGGAGTGGGCCTA | |
| Mixl1 | AAGCCCCAGCTGCCTGTT | NM_031944 |
| | CCCTCCAACCCCGTTTG | (514-576) |
| Nanog | GGCTCTGTTTTGCTATATCCCCTAA | NM_024865 |
| | CATTACGATGCAGCAAATACAAGA | (1899-1980) |
| Oct4 | AGACCATCTGCCGCTTTGAG | NR_002304 |
| | GCAAGGGCCGCAGCTT | (545-600) |
| PAX6 | CTGGCTAGCGAAAAGCAACAG | NM_001604 |
| | CCCGTTCAACATCCTTAGTTTATCA | (865-930) |
| PDGFRα | GATTAAGCCGGTCCCAACCT | NM_006206 |
| | GGATCTGGCCGTGGGTTT | (1989-2053) |
| PDGFRβ | TGGCAGAAGAAGCCACGTT | NM_002609 |
| | GGCCGTCAGAGCTCACAGA | (2135-2197) |
| PROX1 | GATACCACGAGTCTGAGGACCAA | HSU44060 |
| | CGGGTGTGCTGGTGAACA | (2161-2232) |
| PPARγ | TCAGGTTTGGGCGGATGC | NM_138711.3 |
| | TCAGCGGGAAGGACTTTATGTATG | (813-959) |
| Scleraxis | AACACGGCCTTCACTGCGCTG | NM_001080514.1 |
| | CAGCAGCACGTTGCCCAGGTG | (274-396) |
| SOX1 | GCGGTAACAACTACAAAAAACTTGTAA | NM_005986 |
| | GCGGAGCTCGTCGCATT | (1658-1733) |
| SOX2 | TGGTCCTGCATCATGCTGTAG | NM_003106 |
| | AACCAGCGCATGGACAGTTAC | (937-957) |
| SOX6 | GCAGTGATCAACATGTGGCCT | 79 |
| | CGCTGTCCCAGTCAGCATCT | |
| SOX7 | GCTGTCTCCCAGTGGAATGTTC | NM_0031439 |
| | CAAGTCTGTCCCCCCATTAGTT | (1507-1581) |
| SOX9 | GACTTCCGCGACGTGGAC | 80 |
| | GTTGGGCGGCAGGTACTG | |
| SOX17 | AGAGATTTGTTTCCCATAGTTGGATT | NM_022454.3 |
| | TGTTTTGGGACACATTCAAAGCT | (1653-1733) |

1-33. (canceled)

34. A method of producing a mesodermal lineage progenitor cell, the method comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate a stem cell into a mesodermal lineage progenitor cell, preferably a chondro-, osteo-, and/or teno-progenitor cell, preferably a chondro-, osteo-, and/or teno-cyte cell.

35. A method according to claim 34, the method comprising
i) the combined, simultaneous, and/or sequential application of activin and Wnt to a culture of undifferentiated stem cells for a period of time sufficient to differentiate a stem cell into a mesendoderm cell;
followed by ii) the combined, simultaneous, and/or sequential application of one or more factors independently selected from the group consisting of an inhibitor of activin, follistatin and FGF to a culture of cells resulting from i) for a period of time sufficient to differentiate a mesendoderm cell into a mesodermal lineage progenitor cell; and
optionally followed by iii) the combined, simultaneous, and/or sequential application of GDF and NT to a culture of cells resulting from ii) for a period of time sufficient to differentiate a mesodermal lineage progenitor cell into a chondro-, osteo-, and/or teno-progenitor cell.

36. A method according to claim 34, the method comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into a chondroprogenitor cell.

37. A method according to claim 36, wherein the subsequent passaging is in the presence of one or more factors independently selected from the group consisting of FGF, BMP, an inhibitor of activin, and NT, and optionally GDF.

38. A method according to claim 34, the method comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into a osteoprogenitor cell.

39. A method according to claim 38, wherein preferably, the subsequent passaging is in the presence of one or more factors independently selected from the group consisting of FGF, BMP, an inhibitor of activin, and NT, and optionally GDF.

40. A method according to claim 34, the method comprising the combined, simultaneous, and/or sequential application of one or more factors independently selected from the group consisting of activin, Wnt, BMP, FGF, an inhibitor of activin, GDF and NT to a culture of undifferentiated stem cells for a period of time sufficient to differentiate the stem cell into a mesodermal lineage progenitor cell, and subsequent passaging of the mesodermal lineage progenitor cell under conditions suitable to promote its differentiation into a tenoprogenitor cell.

41. A method according to claim 40, wherein the subsequent passaging is in the presence of one or more factors independently selected from the group consisting of FGF, BMP, an inhibitor of activin, and NT, and optionally GDF.

42. A method according to claim 34, the method comprising:
i) the combined, simultaneous, and/or sequential application of one or more factors independently selected from the group consisting of activin, Wnt, FGF and BMP to a culture of undifferentiated stem cells for a period of time sufficient to differentiate the stem cell into a mesendoderm cell;
followed by ii) the combined, simultaneous, and/or sequential application of one or more factors independently selected from the group consisting of BMP, an inhibitor of activin, FGF and NT to a culture of cells resulting from i) for a period of time sufficient to differentiate a mesendoderm cell into a mesodermal lineage progenitor cell; and
optionally followed by iii) combined, simultaneous, and/or sequential application of one or more factors independently selected from the group consisting of FGF, BMP, GDF and NT to a culture of cells resulting from ii) for a period of time sufficient to differentiate the mesodermal lineage into a chondro-, osteo- and/or teno- progenitor cell.

43. A method according to claim 34, the method comprising, in the following order:
i) the combined, simultaneous and/or sequential application of activin and Wnt to a culture of undifferentiated stem cells;
ii) the combined, simultaneous and/or sequential application of activin, Wnt, and FGF to a culture of cells resulting from step i);
iii) the combined, simultaneous and/or sequential application of activin, Wnt, FGF and BMP to a culture of cells resulting from step ii);
iv) the combined, simultaneous and/or sequential application of FGF, BMP, an inhibitor of activin and NT to a culture of cells resulting from step iii);
v) the combined, simultaneous and/or sequential application of FGF, BMP and NT to a culture of cells resulting from step iv); and optionally, comprising the following steps to produce a chondro-, osteo-, and/or teno-progenitor cell from a mesodermal lineage progenitor cell;
vi) the combined, simultaneous and/or sequential application of FGF, BMP,GDF and NT to a culture of cells resulting from step v) above; and
vii) the combined, simultaneous and/or sequential application of FGF, GDF, and NT to a culture of cells resulting from step vi).

44. A method according to claim 43 wherein the cells inhibit the following markers:

| STEP | One or more markers selected from the group consisting of: | |
|---|---|---|
| i | Oct 4 | |
| | Nanog | |
| | MixL (low) | |
| | Bra | |
| | GSc | |
| | Wnt 3 | |
| | N-cad | |
| | E-cad | |
| | Sox 17 (low) | |
| ii | Oct 4 | |
| | MixL (low but preferably higher than in i) | |
| | Bra (high) | |
| | GSc | |
| | E-cad | |
| | Gata 4 | |
| | Sox 17 | |
| iii | As for step ii but preferably lower GSc and/or Gata 4 | |
| iv | low or absent: | Oct 4 |
| | | Nanog |
| | | Gata 4 |
| | | Sox 17 |
| | | Sox 1 |
| | | Pax 6 |
| | | E-cad |
| | Presence of: | MixL, |
| | | PDGF-Rβ |
| | | Flk 1 |
| | | Sox 9 |
| | | Bra (low) |
| v. | As for step iv but low markers are lower, high markers are higher. | |
| | Absence of Bra. | |
| vi | Low or absent: | Nanog |
| | | Oct 4 |
| | | Sox 2 |
| | | E-cad |
| | | Gata 4 |
| | | Sox 17 |
| | | Bra |
| | Presence of: | Sox 9 |
| | | Collagen 2 |
| | | Sox 6 |
| | | CD44 aggrecan |
| | Absence of | Flk 1 |

45. A method according to claim 43, wherein the cells exhibit the following morphological characteristics:
Step i ES like
Step ii Similar to i)
Step iii Initial mesenchymal characteristics
Step iv Initial fibroblastic characteristics, beginning to form whorls
Step vi Clump formation
Step vii Clear aggregates of rounded cells with few cells in between

46. A method according to claim 34, wherein the first stage is performed for a time period sufficient for the resulting mesendoderm cells to show expression of MIXL1, preferably said expression being higher than the expression in the originating stem cells.

47. A method according to claim 34, wherein the second stage is for a time period sufficient for the mesodermal lineage progenitor cells to show expression of brachyury, preferably an increase in expression compared to the mesendoderm cells, and/or Sox9, preferably an increase in expression compared to the mesendoderm cells.

48. A method according to claim 34, wherein the optional third stage is performed for a time period sufficient for the chondro-, osteo-, and/or teno-progenitor cells to show expression of MixL 1 and PDGF-Rβ compared to the mesodermal lineage progenitor cells from which they originate.

49. A method according to claim 34, wherein the method comprises further differentiating any chondro-progenitor, osteo-progenitor and/or teno-progenitor cells.

50. A method according to claim 49, wherein the method may provide for differentiating the chondroprogenitor cells into chondrocyte cells, the osteoprogenitor cells into osteocyte cells and/or the tenoprogenitor cells into tenocyte cells.

51. A method according to claim 34, wherein the cells are grown without the use of feeder cells.

52. A method according to claim 34, wherein the cells are grown in serum-free medium.

53. A method according to claim 34, wherein the stem cell is derived from a stem cell line.

54. A method according to claim 53, wherein the stem cell line is an embryonic stem cell line.

55. A method according to claim 54, wherein the stem cell line is selected from the group consisting of HUES-1, HUES-7, HUES-8, MAN-1 and MAN-2.

56. A method according to claim 53, wherein the stem cell line has one or more of the following characteristics: a) are derived from embryos, preferably embryonic stem cells, preferably at derivation stage d+6 or d+7 or later, b) have a karyotype of 46, c) exhibit paternally imprinted gene expression of H19+, SNRPN+ and/or IGF2+, and d) exhibit stem cell markers independently selected from Nanog, Oct-4, TRA-1-60, TRA-1-81, SSEA-3, and/or SSEA-4.

57. Use of one or more factors independently selected from the group consisting of activin, Wnt, an inhibitor of activin, BMP, FGF, GDF and NT in the combined, simultaneous, and/or sequential application to a culture of undifferentiated stem cells for a period of time sufficient to differentiate a stem cell into a mesodermal lineage progenitor cell, and preferably into a chondro-, osteo- and/or teno-progenitor cell, and more preferably a chondro-, osteo-, and/or teno-cyte cell.

58. A cell culture produced during or by a method according to claim 34.

59. A cell culture selected from the group consisting of:
a cell culture comprising a) undifferentiated stem cells, and b) one or more factors independently selected from the group consisting of activin, Wnt, FGF, and BMP;
a cell culture comprising a) undifferentiated stem cells and mesendoderm cells; and b) one or more factors independently selected from the group consisting of activin, Wnt, FGF, and BMP;
a cell culture comprising a) mesendoderm cells; and b) one or more factors independently selected from the group consisting of BMP, FGF, NT and an inhibitor of activin;
a cell culture comprising a) mesendoderm and mesodermal lineage progenitor cell; and b) one or more factors independently selected from the group consisting of BMP, FGF, NT and an inhibitor of activin;
a cell culture comprising a) mesodermal lineage progenitor cells; and b) one or more factors independently selected from the group consisting of FGF, BMP, GDF and NT;
a cell culture comprising a) mesodermal lineage progenitor cells, chondro-, osteo-and/or teno- progenitor cells; and b) one or more factors independently selected from the group consisting of FGF, BMP, GDF and NT; and
a cell culture comprising a) a chondro-, osteo-, and/or teno-progenitor cell, and b) one or more factors independently selected from the group consisting of FGF, BMP, an inhibitor of activin, NT and GDF; and a chondro-, osteo-, and/or teno-cyte cell.

60. A cell culture according to claim 59 for use in the treatment of a bone, tendon and/or cartilage defect in a subject.

61. A cell produced by a method of claim 34.

62. A matrix comprising one or more cells according to claim 61.

63. A kit of parts comprising, in separate containers, one or more factors independently selected from the group consisting of activin, Wnt, BMP, FGF, an inhibitor of activin, GDF, NT and a culture of undifferentiated stem cells, and optionally instructions for use, and/or a protocol detailing the method of claim 34.

64. A method of producing a chondro-, osteo-, and/or teno-progenitor cell from a mesodermal lineage progenitor cell, the method comprising:
a) the combined, simultaneous and/or sequential application of one or more factors independently selected from FGF, BMP, GDF and NT to a culture of mesodermal lineage progenitor cells; and
b) the combined, simultaneous and/or sequential application of one or more factors independently selected from FGF, GDF, and NT to a culture of cells resulting from step a).

65. A method according to claim 64, optionally preceded by one or more of steps i) to v) of claim 43.
